(19) United States
(12) Patent Application Publication
(10) Pub. No.: US 2018/0315488 A1
(43) Pub. Date: Nov. 1, 2018

(54) Emergency Room Medical Triage, Diagnosis, and Treatment

(71) Applicant: Telemedco Inc., Bronx, NY (US)

(72) Inventors: Altagracia Miranda, Miami Beach, FL (US); Barry Solomon, Larchmont, NY (US)

(21) Appl. No.: 15/962,956

(22) Filed: Apr. 25, 2018

Related U.S. Application Data
(60) Provisional application No. 62/489,774, filed on Apr. 25, 2017.

(51) Int. Cl. G16H 10/20; G16H 10/60; G16H 50/20; G16H 40/20

(52) U.S. Cl. CPC G16H 10/20; G16H 10/60; G16H 15/00; G16H 40/20; G16H 50/20

(57) ABSTRACT

Disclosed are systems and methods for triaging, diagnosing, and treating patients. A question-answering device can receive input from a patient regarding the malady the patient is seeking treatment for. The question-answering device can determine based on the input from the patient a likely malady and a recommended course of treatment to cure the malady.

### CROSS REFERENCE TO RELATED PATENT APPLICATION

This application claims priority to U.S. Provisional Application No. 62/489,774, filed Apr. 25, 2017, which is herein incorporated by reference in its entirety.

### FIELD OF INVENTION

The present invention relates to a question-answering computing system capable of utilizing advanced natural language processing, information retrieval, knowledge representation, automated reasoning, and machine learning technologies.

### BACKGROUND

Emergency rooms are essential in providing urgent care to patients. However, in the past two decades the number of emergency rooms dropped by more than 25%, while the patient visits continue to rise. For example, in 2011 there were 136.3 million visits to emergency rooms in the United States, which represents a rate of 421 emergency room (ER) admissions per 1000 population of the US. It was shown that from all ER admissions 1.2% of these admissions required immediate care, 10.7% required emergent care, 42.3% required urgent care, while 45.7% required lower levels of care or none at all. The average time spent in the emergency room ranges from 3 to 5 hours, while in some states the average time spent in the emergency room can rise to 7 hours. The continuous increase in medical costs coupled with fewer resources represents a growing challenge of efficiently and thoroughly caring for patients in emergency rooms.

Emergency department triage and patient care are particularly difficult, even for well-trained and experienced medical professionals when they operate at or near capacity and work with a large number of varying conditions. It is imperative for medical professionals to determine an appropriate decision tree based on a variety of protocols approved by the American College of Emergency Physicians. Additionally, most hospital systems are designed to record and store patient records, not provide care. Accordingly, the computer systems in use in hospitals are unable to triage, diagnose, and treat patients. Thus, there is a need for improved methods and systems capable of providing highly professional, yet efficient, care to patients in an emergency room regardless of the patient's situation. These and other shortcomings are addressed by the methods and systems described herein.

### SUMMARY

It is to be understood that both the following general description and the following detailed description are exemplary and explanatory only and are not restrictive. Provided are methods and systems for triaging, diagnosing, and treating patients in an emergency room setting to ensure an efficient and thoroughly treatment.

In one aspect, a method is disclosed. The method comprises receiving, by a computing device from a patient, information related to malady of the patient. The method also comprises retrieving, by the computing device from a database storing medical histories of a plurality of patients, a medical history of the patient. The method further comprises determining, by the computing device based on the received information and the medical history of the patient, a first question to ask the patient. The method additionally comprises providing, by the computing device, the first question to the patient. The method comprises receiving, by the computing device, from the patient a first answer. The method also comprises determining, by the computing device based on the received first answer, the received information and the medical history of the patient, a diagnosis of the patient. Additionally, the method comprises determining, by the computing device based on the diagnosis of the patient, a treatment plan for the patient.

In another aspect, an apparatus is disclosed that comprises one or more processors and a memory storing processor executable instructions that, when executed by the one or more processors, cause the apparatus to receive from, a patient, information related to a malady of a patient. The processor executable instructions further cause the apparatus to retrieve, from a database storing medical histories of a plurality of patients, a medical history of the patient. The processor executable instructions further cause the apparatus to determine, based on the received information and the medical history of the patient, a first question to ask the patient. The processor executable instructions further cause the apparatus to provide the first question to the patient. The processor executable instructions further cause the apparatus to receive from the patient a first answer. The processor executable instructions further cause the apparatus to determine, based on the received first answer, the received information and the medical history of the patient, a diagnosis of the patient. The processor executable instructions further cause the apparatus to determine, by the computing device based on the diagnosis of the patient, a treatment plan for the patient.

In another aspect, a computer readable medium is disclosed that comprises computer executable instructions configured to cause a computing device to receive, from a patient, information related to a malady of the patient. The computer executable instructions further cause the computing device to retrieve, from a database storing medical histories of a plurality of patients, a medical history of the patient. The computer executable instructions further cause the computing device to determine, based on the received information and the medical history of the patient, a first question to ask the patient. The computer executable instructions further cause the computing device to provide the first question to the patient. The computer executable instructions further cause the computing device to receive from the patient a first answer. The computer executable instructions further cause the computing device to determine, based on the received first answer, the received information and the medical history of the patient, a diagnosis of the patient. The computer executable instructions further cause the computing device to determine, by the computing device based on the diagnosis of the patient, a treatment plan for the patient.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems.

FIGS. 1A-1B illustrate exemplary systems.

FIG. 2 is a diagram showing an example computing system for providing emergency room triage, diagnosis, and treatment.

FIG. 3 is a diagram showing an example computing device.

FIG. 4 is a flowchart of an example method.

FIG. 5 is a diagram showing the alternate pathways for a chest pain complaint triage.

FIG. 6 is a diagram showing ED observation protocol for hemodynamically stable atrial fibrillation.

FIG. 7 is a chart showing clinical guideline for undifferentiated “dizziness.”

FIG. 8 is a chart showing the approach to non-traumatic headache in an adult (18+ years).

FIG. 9 is a chart showing DKA (Diabetic Ketoacidosis)/HHS (Hyperosmolar Hyperglycemic State) pathways in an adult.

FIG. 10 is a chart showing Adult ED glycemic control protocol.

FIG. 11 is a chart showing insulin preparations.

FIG. 12 is a table showing guideline evidence of carbon monoxide poisoning.

FIG. 13 is a chart showing a guideline for frostbite treatment.

FIG. 14 is a chart showing management of massive upper GI bleed.

FIG. 15 is a chart showing febrile sickle cell or asplenic patient clinical practice guideline emergency department—initial treatment.

FIG. 16 is a chart showing febrile pediatric oncology patient clinical practice guideline emergency department—initial treatment.

FIG. 17 is a chart showing the treatment for a 1^{st} or 2^{nd} episode of CDAD (Clostridium Difficile-Associated Diarrhea).

FIG. 18 is the clinical practice guideline for bronchiolitis.

FIG. 19 is a table regarding the treatment of Lyme Disease.

FIG. 20 is a chart showing non-traumatic myelopathy and Cauda Equina Syndrome protocol.

FIG. 21 is a table showing spine triage guidelines (non-trauma).

FIG. 22 is a chart showing a guideline for pediatric fever.

FIG. 23 is a chart showing a guideline for pediatric minor blunt head trauma.

FIG. 24 is a table showing pediatric status epilepticus evidence table.

FIG. 25 is a chart showing the MMC asthma exacerbation guideline.

FIG. 26 is a chart showing high risk pulmonary embolism.

FIG. 27 is a chart showing non-high risk pulmonary embolism.

FIG. 28 is a chart showing minor blunt head trauma algorithm.

FIG. 29 is a chart showing MMC guideline for reversal of Warfarin-associated CNS (Central Nervous System) Hemorrhage.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

The term “triage,” as used herein, refers to emergency room proceedings where the patients are sorted based on criticality ratings. The criticality ratings are called triage levels. The methods and systems disclosed herein employ a five-tier triage scale consisting of levels I, II, III, IV, and V, which are referred to, respectively, as immediate, emergent, urgent, semi-urgent, and non-urgent. Level I conditions are life threatening and require immediate medical attention and intervention. Level II conditions are potentially life threatening and require emergent medical attention. Level III conditions may develop into a serious problem unless urgent medical evaluation and intervention is received. Level IV conditions are semi-urgent and may benefit from intervention to prevent deterioration or complications. Finally, Level V conditions are non-urgent conditions that are minor and could be delayed or deferred to other areas of the health care system with little risk of significant deterioration.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

As used herein the term “receiving” is not intended to be used in any restrictive manner. It is understood that the term encompasses a physical receiving of a stated matter, an electronic receiving of a stated matter, a verbal or a written receiving of a stated matter, or any combination thereof. In some cases, the particular term utilized may be dependent on the context in which it is used. For example, receiving information from the patient or caregiver may also refer to a verbal conversation with the patient or caregiver. In another example, receiving health records may also refer to an electronic connection with an entity hosting health records and electronically transferring the information from the hosting entity to a computing system.

As used herein the term “worldwide web” refers to an information space where documents and other web resources are identified by uniform resource indicators (URIs) such as uniform resource locators (URLs), interlinked by hypertext links, and can be accessed via the internet. As an example, the term “worldwide web” can include at least a portion of the Internet.

Note that in various instances this detailed disclosure may refer to a given entity performing some action. It should be understood that this language may in some cases mean that a system (e.g., a computer) owned and/or controlled by the given entity is actually performing the action.

Typically, when a patient seeks medical care from a healthcare provider, a nurse or a medical technician asks the patient several questions about the patient's condition to evaluate the patient. The nurse or the medical technician may or may not take the vital signs of the patient. Based on this initial evaluation of the patient's symptoms, and possibly vital signs, the medical professional then makes a decision about the criticality of the patient's condition and assigns a criticality or triage rating to each patient. Patients are then prioritized for receiving medical attention based on the medical professional's assigned rating.

Currently, the decision regarding the criticality of the patient's condition can never be better than the experience and training level of the attending medical professional. If the attending medical professional is not well trained, is inexperienced, or for some other reason fails to ask the right questions, or if the patient is suffering from a condition that is rarely seen or a condition that exhibits symptoms that appear less severe than the actual critical nature of the patient's malady, the medical professional can easily make an error in assigning the criticality to the patient's condition.

The present methods and systems may be understood more readily by reference to the following detailed description of preferred embodiments and the examples included therein and to the Figures and their previous and following description.

The disclosed system is a chief complaint-based, algorithm-driven triage system. The system may comprise a computing device in communication with a hospital database and a question-answering processing system. The computing device is in a communication with a patient and/or caregiver and a medical care professional. In some aspects, the communication is a direct communication. In other aspects, the communication can be written or verbal. In yet other aspects, the communication is a virtual communication. It is understood that the term “virtual communication” as used herein can comprise a communication using a text-based, a video, or an audio communication. As examples, the communication can be any real-time or substantially real-time communication. As particular examples, electronic communications can take the form of video chat (e.g., using videotelephony standards such as International Telecommunications Union (ITU) H.320 public switched telephone network (PSTN) standard, ITU H.264 scalable video coding standard, ITU V.80 videoconferencing standard, and/or the like), audio chat (for example, via telephone, voice over Internet Protocol, and/or the like), text-based communications (for example, using standards such as real-time text, Session Initiation Protocol (SIP) messaging, Extensible Message and Presence Protocol (XMPP) messaging, and/or the like), and/or combinations thereof. It is further understood that when a computing device is in communication with a patient and/or a caregiver, this communication includes a direct communication with a patient, a direct communication with a patient's caregiver, or any person knowledgeable of the patient's state of health.

In some aspects, the medical health care professional can be a paramedic, a nurse, a doctor, a physician assistant, a billing specialist, hospital receptionist, or any combination thereof.

The disclosed system further comprises a Question-Answering (QA) device. The QA device of the disclosed system can comprise one or more processors. The QA device of the disclosed system can comprise an interface capable of communicating with other computing devices. The QA device can comprise a cluster of computer processors that can be used to execute natural language processing, information retrieval, automated reasoning, and/or machine learning processes to execute the processes described herein. It is understood that any processing devices used in the Question-Answering branch of computer science can be used in the described systems and methods. As a particular example, IBM Watson™ is a QA computing system built to apply advanced natural language processing, information retrieval, knowledge representation, automated reasoning, and machine learning technologies to the field of open domain question-answering. As such, IBM Watson™ is an exemplary question-answering processing device capable of performing the described methods and utilized in the disclosed system.

In certain aspects, the disclosed system further comprises a health care provider database that is in communication with the computing device. In some aspects, the health care provider database can comprise one or more of a health record database, a health care database, an external research database, or any combination thereof. The health care provider database can comprise a health record of a plurality of patients of the health care provider. In yet other aspects, the health record of the patient can be kept by an entity other than the health care provider. In these aspects, the computing device and/or question-answering processing device can be in communication with the specific entity hosting the patient health record.

It is understood that the disclosed system comprises an interface that allows it to be integrated with any existing hospital computer systems, thereby allowing easy access to any information kept by the health care provider. In some aspects, the interface can further be used to communicate with the patient and/or caregiver and the medical care professional. Still further, the interface can be used to facilitate communication among the patient and/or caregiver, the medical care professional, and the existing hospital computer systems.

In some aspects, the computing device of the present disclosure is configured to perform the step of receiving information from the patient or the caregiver. This information can be presented by the patient, the caregiver, or any person knowledgeable of the patient's symptoms or state of health. It is understood that rarely does a patient come to a health care provider with a diagnosis in hand, but rather they present with a symptom or group of symptoms and the health care professional needs to determine a diagnosis based on symptoms of the patient. The information can comprise a chief complaint or a symptom or a group of symptoms of the patient. In yet other aspects, the information comprises an age and a sex of the patient. In still further aspects, the information comprises a past medical history if available. The computing device is configured to collect this information to provide it to the question-answering processing device for further processing. Further, the computing device is configured to communicate with the health care provider database to determine any previous medical records to facilitate diagnosing the patients symptoms.

The computing device is further configured to communicate with the patient or the caregiver. In some aspects, the computing device is capable of questioning the patient and/or caregiver using a set of scripted questions to receive answers. In some aspects, the step of questioning can be conducted in any language chosen by the patient or caregiver. The computing device can be configured to provide a question in an audible form of the patient. For example, the computing device can be configured to provide questions orally in English in response to the patient or caregiver selecting English as the language the patient or caregiver desires to communicate in. It is further understood that the step of questioning can be conducted, verbally, written or virtually, or in any combination thereof. In one aspect, the computing device facilitates the QA processing device communicating with the patient. That is, the QA processing device communicates with the patient via the computing device. While the computing device and the QA processing device are described as separate devices for ease of explanation, a person skilled in the art would appreciate that the computing device can comprise the QA processing device.

In certain aspects, the step of questioning is adjusted to accommodate people having hearing, speech, or vision disabilities. In certain aspects, the step of questioning includes a set of scripted questions that has been developed according to the American College of Emergency Physicians standards. In certain aspects, the questions provided by the computing device depend on the chief complaint given by the patient or the caregiver. For example, if the patient indicates that their arm hurts, the computing device will follow up with additional questions that are associated with arm injuries to better determine the exact problem of the patient. In yet other aspects, the computing device can adjust the set of question depending on the answers received from the patient or caregiver. For example, if the patient indicates their arm hurts, the computing device will not ask questions about unrelated body parts (i.e., the patient's foot).

In other aspects, the computing device is capable of analyzing the answers received from the patient and/or caregiver to determine the nature of a patient risk. For example, the computing device can provide the answers received from the patient and/or caregiver to the QA processing device for analysis. The QA processing device can comprise a question classifier module that can determine a type of question and a corresponding type of answer. The QA processing device may comprise a multi-agent QA processing device that comprises a plurality of agents having access to research directed to a specific domain of knowledge. Each agent can provide an answer to a received question taking into account knowledge specific to the agent. The answers from each agent can be provided to a meta-agent, which controls cooperation between QA agents and chooses one or more relevant answers from among the answers received at the meta-agent. In particular, the QA processing device can parse questions into different keywords and sentence fragments in order to find statistically related phrases. The QA processing device can execute one or more language analysis algorithms to determine a candidate answer based on the question, the determined keywords, the statistically related phrases, and/or combinations thereof. In some aspects, a candidate answer that is independently determined by multiple algorithms can be weighted such that the QA processing device is more likely to determine that the candidate's answer is correct. Once the QA processing device has a small number of candidate answers, the QA processing device can check each candidate answer against a database. The QA processing device can then provide the answer to the computing device, which in turn provides the answer to the patient.

As a further example, for healthcare related questions, natural language, hypothesis generation, and evidence-based learning capabilities of a plurality of QA processing devices can be used to contribute to clinical decision support systems for use by health care professionals. To aid the health care professionals in treatment of patients, once a health care professional has posed a query to the QA processing device describing symptoms and other related factors, the QA processing device can parse the input to identify important pieces of information. Once the QA processing device has identified important pieces of information, the QA processing device can then determine facts relevant to patient medical and hereditary history. Based on the relevant history and the identified important pieces of information, the QA processing device can examine available data sources to form and test hypotheses and provide a list of individualized, confidence-scored recommendations of possible diagnoses related to the symptoms that the patient is presenting. The QA processing device can access resources that include treatment guidelines, electronic medical record data, notes from physicians and nurses, research materials, clinical studies, journal articles, and patient information.

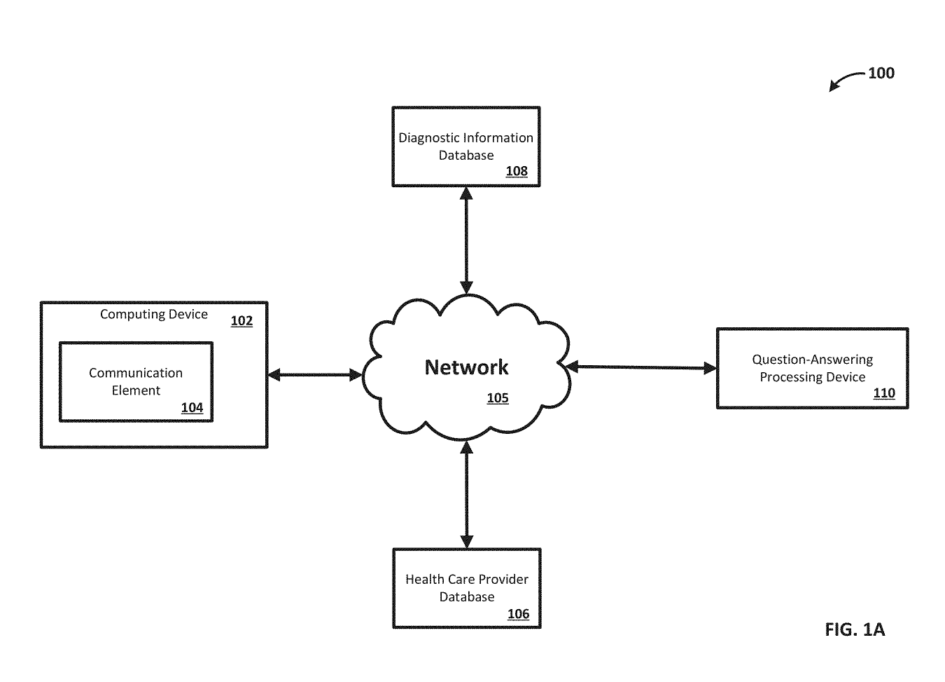

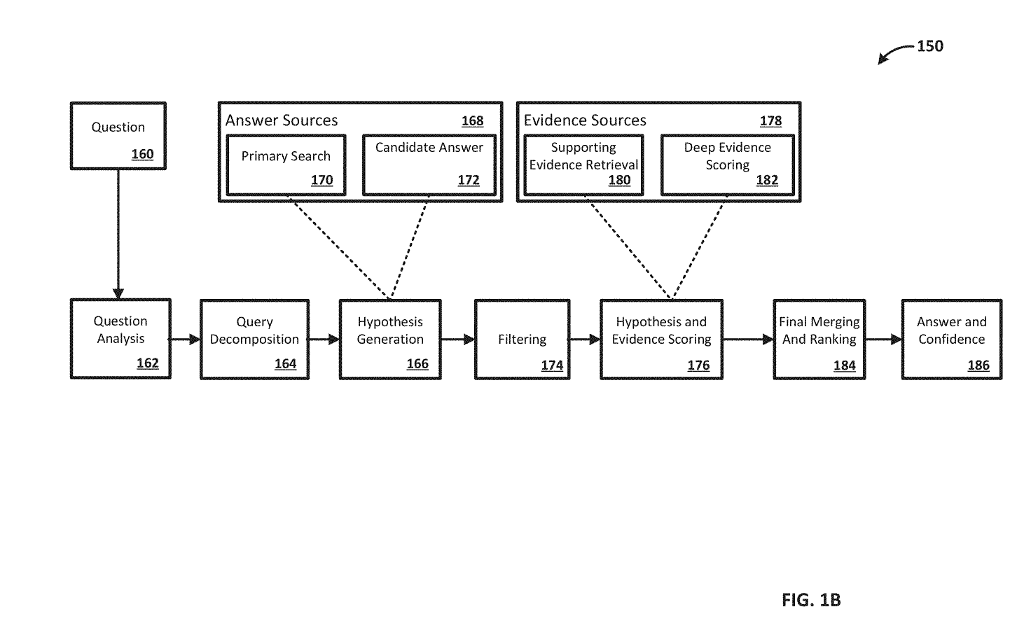

FIG. 1A shows an exemplary system 100 in which the present methods and systems may operate. The example system 100 comprises one or more computing devices 102, a healthcare provider database 106, a diagnostic information database 108, and a Question-Answering (QA) processing device 110, that can be in communication via a private and/or public network 105 such as the Internet, a local area network, and/or a mesh network. Those skilled in the art will appreciate that the present methods may be used in systems that employ both digital and analog equipment. One skilled in the art will appreciate that provided herein is a functional description and that the respective functions may be performed by software, hardware, or a combination of software and hardware.

The computing device 102 can be an electronic device such as a computer, a smartphone, a laptop, a tablet, or other device capable of communicating with a user. As an example, the computing device 102 can comprise a communication element 104 for providing an interface to a user to interact with the computing device 102, the healthcare provider database 106, the diagnostic information database 108, and/or the QA processing device 110. The communication element 104 can be any interface for presenting and/or receiving information to/from the user. For example, the communication element 104 can be an interface that allows the user to provide information to the computing device 102 regarding symptoms the user is experiencing. Additionally, the communication element 104 can provide information to the user, such as a diagnosis based on the user's symptoms. An example interface may be communication interface such as a web browser (e.g., Internet Explorer, Mozilla Firefox, Google Chrome, Safari, or the like). Other software, hardware, and/or interfaces can be used to provide communication between the user and one or more of the computing device 102, the health care provider database 106, the diagnostic information database 108, and/or the QA processing device 110. As an example, the communication element 104 can communicate with the health care provider database 106 to retrieve the user's medical history, if any, that is stored on the health care provider database 106. As a further example, the communication element 104 can transmit information to the QA processing device 110 in order for the QA processing device 110 to determine a diagnosis for the user based on the user's symptoms and medical history.

The health care provider database 106 can be a database storing any information associated with a health care provider such as patient medical records and health care capabilities of the health care provider. For example, if the health care provider is a hospital, the health care provider database 106 can store information related to any patient's medical history that has visited the hospital. Further, the health care provider database 106 can store information related to the capabilities of the health care provider. As an example, the health care provider database 106 can store information related to all nurses or doctors currently working for the health care provider including specialties and whether the nurse and/or doctor is currently busy, any medications that are immediately accessible to a health care professional, the number of hospital rooms and/or beds available or in use, and any protocols the health care provider has. For example, the health care provider may have specific procedures for handling specific types of emergencies and/or communicable diseases.

The computing device 102 and/or the QA processing device 110 can access and use the information stored in the health care provider database 106 to provide a diagnosis and/or treatment for a patient based on the capabilities of the health care provider. For example, once the computing device 102 and/or the QA processing device 110 determines a diagnosis for the patient, the computing device 102 and/or the QA processing device 110 can provide a recommending course of treatment based on the capabilities of the health care provider. As an example, if the course of treatment involves administering a specific medicine, the computing device 102 and/or the QA processing device 110 can determine if the health care provider has the medicine in stock, and if the medicine is available, the computing device 102 and/or the QA processing device 110 can notify a health care professional to retrieve the medicine and administer the medicine to the user. As another example, if the diagnosis is related to a rare disease and the health care provider does not have a specialist capable of properly treating the disease, the recommended course of treatment may be to transfer the patient to a different health care provider to treat the patient. As further example, if the recommended course of treatment involves medicine the health care provider does not have in stock, the recommend course of treatment might include ordering more of the medicine and/or transferring the patient to another health care provider. In this manner, the computing device 102 and/or the QA processing device 110 can use the information stored in the health care provider database 106 to provide a recommended course of treatment for the user.

The diagnostic information database 108 can be a database storing any information related to diagnosing and determining a recommended course of treatment for a malady of a patient. For example, the diagnostic information database 108 can store information from the Association of American Medical Colleges to determine treatments and diagnosis of patients. Further, the diagnostic information database 108 can store questions that a health care profession would ask a patient to determine a diagnosis for a patient. For example, if a patient is complaining of an arm, the questions may pertain to determining whether the patient has a broken arm. The computing device 102 and/or the QA processing device 110 can use the diagnostic information database 108 to determine what questions to ask a patient based on the presented symptoms, determine a diagnosis for the patient based on the responses received, and determine a recommended course of treatment.

The QA processing device 110 can communication with the computing device 102, the health care provider database 106, and/or the diagnostic information database 108. The QA processing device can be configured to perform natural language processing. For example, the QA processing device can receive a patient's response and interpret response to determine follow up questions or a diagnosis of the patient. The QA processing device can receive data from the computing device 102 that comprises information related to a patients input regarding symptoms the patient has. The QA processing device 110 can generate one or more questions based on the received data and provide the questions to the computing device 102. The computing device 102 in turn can present the questions to the patient in order to elicit a response from the patient.

The QA processing device 110 can use machine learning to improve the function of the QA processing device. For example, the QA processing device 110 can communicate with the health care provider database 106 to determine the capabilities and records of the health care provider to allow the QA processing device 110 to better diagnose patients of the health care provider. Further, the QA processing device 110 can communicate with the diagnostic information database 108 to determine proper procedures for diagnosing a patient. For example, the diagnostic information database 108 can contain guidelines for the proper diagnosis of an ailment based on the symptoms a patient is presenting, as well as the proper treatment for the ailment. Further, the diagnostic information database 108 can contain guidelines for the questions to ask a patient in order to determine the ailment the patient has. The QA processing device 110 can use the information stored in the diagnostic information database 108 to determine the proper questions, diagnosis, and treatment for the patient based on the symptoms the patient is presenting. In this manner, the QA processing device 110 is capable of learning from the health care provider database 106 and/or the diagnostic information database 108 to facilitate diagnosing the ailment of a patient based on the symptoms and answers the patient provides.

FIG. 1B shows an exemplary method 150 for a QA processing device 110. The QA processing device 110 can receive a question at block 160. The question can be received via, for example, an electronic communication, a voice recording, physical speech, and or the like. For example, the computing device 102 of FIG. 1A can provide the question to the QA processing device 110. In some aspects, the question can be received as a natural language question processed via a natural language processing module (not shown). In other aspects, the question format can be predefined, and the question can be received as a list of properties associated with the questions. For example, where the question relates to potential diagnoses of a patient, the question can be received as one or more symptoms or other properties of the patient (e.g., demographic information, presented abnormalities, medical history, and/or the like). While the method is described in terms of receiving a question, a person skilled in the art would appreciate a similar method can be performed in response to receiving an answer to a question presented to the user.

At block 162, the received question can be analyzed. For example, the question can be classified. As another example, the question can be analyzed to determine keywords or phrases within the question that can impact a query based on the question. The processing can further comprise determining statistical relationships among the one or more keywords and/or the one or more sentence fragments.

At block 164, the received question can be decomposed into one or more keywords and/or sentence fragments. As an example, a question such as “why does my left arm hurt” can be broken down into important keywords to allow for easier processing of the question. That is, a query can be based on a keyword or phrase, rather than an entire question to facilitate finding an answer to the question.

At block 166, a hypothesis can be generated based on the decomposed question. For example, a search can be performed of one or more answer sources 168 based on the decomposed question in order to determine an answer for the question. In one example, a primary search 170 can be performed in the answer sources 1689 to determine a candidate answer 172. In this manner, the QA processing device 110 can determine a candidate answer 172 based on the answer sources 168. While a single hypothesis generation 166 is shown for ease of explanation, a person skilled in the art would appreciate that any number of hypothesis generation 166 may occur and that each hypothesis generation 166 may have an respective primary search 170 of the answer sources 168. In this manner, the QA processing device 100 is capable of determining multiple candidate answers 172 for multiple hypothesis generations 166.

At block 174, the candidate answer 172 may be filtered 174 to remove obviously incorrect answers prior to hypothesis scoring. For example, if the question is “why does my arm hurt?,” the filtering 174 may remove candidate answers 172 that relate to foot pain as those answers would be irrelevant for answering why the patient's arm hurts.

At block 176, the hypothesis scoring 176 scores the candidate answer 172 based on one or more evidence sources 178. As an example, the hypothesis scoring 176 can retrieve supporting evidence 180 from the one or more evidence sources. Based on the retrieved supporting evidence 180, the hypothesis can be scored. In an aspect, the hypothesis can be scored based deep evidence scoring 182 which can rate various types of evidence based on weight assigned to the evidence. For example, if the evidence source 178 is a well-respected medical journal, the medical journal may receive a high rating from the deep evidence scoring 182. Alternatively, if the evidence source 178 is from an only blog from a person without a medical degree, the blog may receive a low rating from the deep evidence scoring 182. In this manner, the QA processing device is capable of differentiating between a strong source of supporting evidence and a weak source of supporting evidence.

At block 184, the hypothesis, the candidate answer 172, and the supporting evidence 180 can be merged and ranked based on trained models. The final ranking of the candidate answers can be generated according to the likelihood that the candidate answers is correct. For example, the hypothesis that has a candidate answer 172 with the most supporting evidence 180 can be ranked highly versus a hypothesis that has a candidate answer 172 with no supporting evidence 180. In this manner, the QA processing device is capable of ranking the candidate answers based on the supporting evidence 180. Further, the QA processing device can determine a confidence for each candidate answer 172 based on the answer sources 168 and the evidence sources 178. For example, the answer can comprise a percentage, a fraction, or any other score indicating a confidence that the answer is correct.

At block 186, an answer can be communicated to the question asker. The answer can include a confidence score which indicates the confidence in the answer being correct. In an aspect, the answer can comprise the candidate answer 172 that has the highest confidence score. In another aspect, a plurality of answers can be provided each with a respective confidence score so as to allow the user receiving the answer to make the final determination as to what the correct answer is.

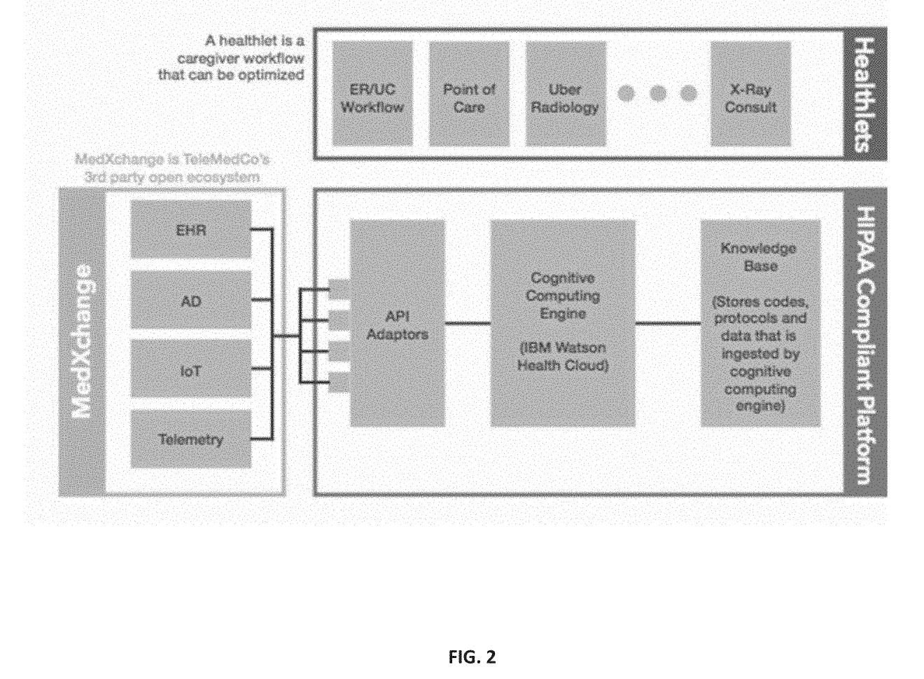

FIG. 2 shows a schematic illustration of a computing system using the QA processing device. In an aspect, a cognitive computing engine comprising the QA processing device 110 can be in communication with a knowledge base (for example, a corpus or database comprising the one or more answer sources and the one or more evidence sources discussed in FIG. 1). The cognitive computing engine can further communicate with one or more application program interfaces (APIs). The cognitive computing engine, knowledge base, and the APIs can collectively form a Health Insurance portability and Accountability Act (HIPAA) compliant platform for use with a computing device. The HIPAA compliant platform can communicate, via the one or more APIs, with one or more third party computing systems. For example, the HIPAA compliant platform can communicate with an electronic health record server, various telemetry devices, various Internet of Things devices (for example, one or more Internet-connected devices such as smart devices, internet-connected diagnostic tools (for example, blood pressure monitors, thermometers, intravenous fluid pumps, and/or the like)), and/or combinations thereof. The HIPAA compliant platform, together with the third party computing systems, can be used to help optimize one or more caregiver workflows, also known as Healthlets.

It is understood that in some aspects, the patient is observed by the medical professional before the patient is in communication with the computing device 102 and the QA processing device 110. In such aspects, the observations obtained by the medical professional are recorded into the computing device 102. It is understood that the observations obtained by the medical professional and recorded into the computing device 102 can comprise vital signs, lab analysis, imaging, physical observations, or any combination thereof. In these aspects, the QA processing device 110 that is in communication with the computing device 102 can access the recorded observation and adjust the step of questioning based on the recorded observations. For example, the recorded observation can be stored within the health care provider database 106.

In other aspects, the computing device 102 is configured to receive from the health care provider database 106, an electronic health record of the patient, if present. In the aspects where the electronic health record of the patient is not present, the computing device 102 can be configured to communicate with an entity that hosts the patient's health record, or initiate a new health record, or a combination thereof.

In yet other aspects, the computing device 102 is further configured to provide the electronic health record, if present, to the QA processing device 110 for processing based on the electronic health record if present and answers provided by the patient to determine a protocol comprising a treatment plan. In yet other aspects, the QA processing device 110 can further process observation provided and recorded by the medical professionals to determine a protocol comprising a treatment plan. In some aspects, the treatment plan comprises assigning a triage level of the patient. In yet other aspects, the treatment plan comprises conducting a test. It is understood that the test can comprise any test known in the art, for example and without any limitations the testing can comprise blood lab analysis, imaging testing, such as MRI, PET, CT, X-ray, etc., electrocardiogram, encephalogram, stress testing, etc. Further, the treatment plan can include medications and instructions for the patient as to how to treat the malady of the patient. In yet other aspects, the treatment plan can comprise a consultation with a specialist. In still further aspects, the treatment plan can comprise any or all mentioned above procedures.

The QA processing device 110 can generate a report comprising the information provided by the patient and/or caregiver, the electronic health record if present, and the answers provided to the QA processing device 110 by the patient and/or caregiver, the treatment plan, or any combination thereof. In still further aspects, the computing device 102 is configured to receive this report from the QA processing device 110 and communicate the report to the medical health care professional.

In some aspects, the QA processing device 110 is in communication with the worldwide web via the network 105. In these aspects, the QA processing device 110 can further analyze the data present in the worldwide web to provide an alternative protocol comprising an alternative treatment plan. In still further aspects, where the alternative protocol comprising the alternative treatment plan is present, the QA processing device 110 and the computing device 102 are capable of comparing the treatment plan and the alternative treatment plan to provide a comparison report for the medical professional review.

It is understood that the disclosed system is capable of spotting potentially life threatening clinical conditions based on the information provided by the patient and/or caregiver, medical professional observations, answers provided by the patient and/or caregiver to the questions asked by the QA processing device 110, the health care records stored in the health care provider database 106, and to determine a treatment plan for the patient. The treatment plan may include assigning a triage level to the patient.

It is further understood that any step, the treatment plan assigned by the QA processing device 110 is reviewed by a medical professional. In the aspects, where the triage level is assigned, the medical professionals can override the system, if medical professionals believe that a patient's condition is more critical than the triage level assigned by the system. It is understood that the system can always be overridden to require a higher and/or lower triage level for a patient. In one aspect, as a conservative measure, the QA processing device 110 cannot be overridden to assign a lower triage level for a patient, i.e. a triage level that would indicate the patient is not as critical as the triage level that was assigned to the patient by the system in order to ensure patient safety.

The QA processing device 110 can provide a final report comprising recommendation to admit or discharge the patient. In still further aspects, the QA processing device 110 can provide a prescription for medications for the medical professional signature. In yet other aspects, the QA processing device 110 can generate a report for billing purposes.

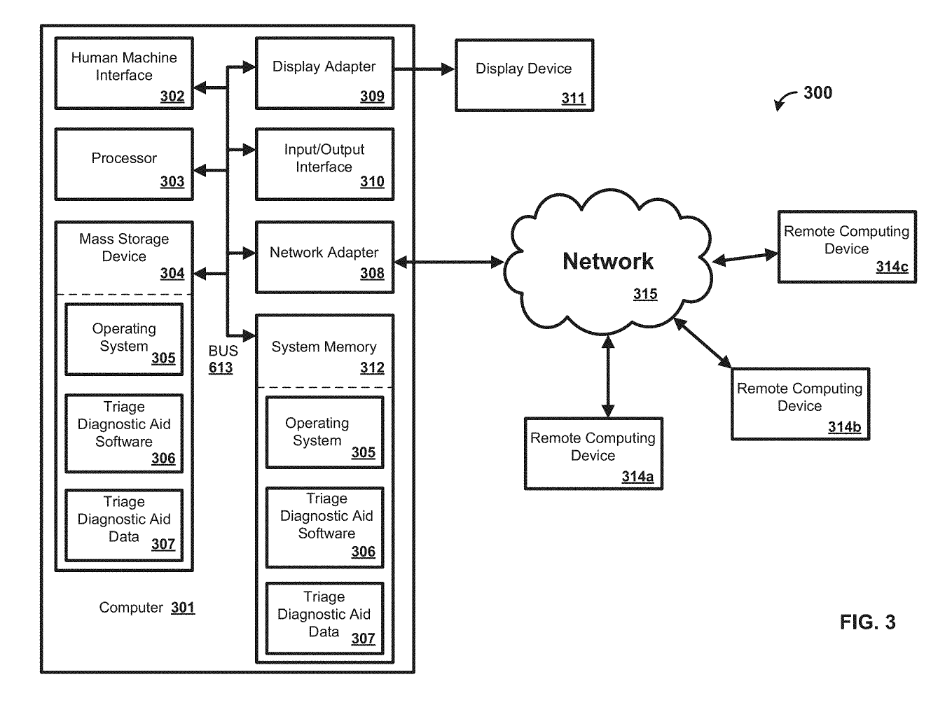

In an exemplary aspect, the methods and systems can be implemented on a computer 301 as illustrated in FIG. 3 and described below. By way of example, the computing device 102, the health care provider database 106, the diagnostic information database 108, and/or the QA processing device 110 can be a computer 301 as illustrated in FIG. 3. Similarly, the methods and systems disclosed can utilize one or more computers to perform one or more functions in one or more locations. FIG. 3 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 301. The components of the computer 301 can comprise, but are not limited to, one or more processors 303, a system memory 312, and a system bus 313 that couples various system components including the one or more processors 303 to the system memory 312. The system can utilize parallel computing.

The system bus 313 represents one or more of several possible types of bus structures, including a memory bus or memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 313, and all buses specified in this description can also be implemented over a wired or wireless network connection and each of the subsystems, including the one or more processors 303, a mass storage device 304, an operating system 305, triage diagnostic aid software 306, triage diagnostic aid data 307, a network adapter 308, the system memory 312, an Input/Output Interface 310, a display adapter 309, a display device 311, and a human machine interface 302, can be contained within one or more remote computing devices 314a,b,c at physically separate locations, connected through buses of this form, in effect implementing a fully distributed system.

The computer 301 typically comprises a variety of computer readable media. Exemplary readable media can be any available media that is accessible by the computer 301 and comprises, for example and not meant to be limiting, both volatile and non-volatile media, removable and non-removable media. The system memory 312 comprises computer readable media in the form of volatile memory, such as random access memory (RAM), and/or non-volatile memory, such as read only memory (ROM). The system memory 312 typically contains data such as the triage diagnostic aid data 307 and/or program modules such as the operating system 305 and the triage diagnostic aid software 306 that are immediately accessible to and/or are presently operated on by the one or more processors 303.

In another aspect, the computer 301 can also comprise other removable/non-removable, volatile/non-volatile computer storage media. By way of example, FIG. 3 illustrates the mass storage device 304 which can provide non-volatile storage of computer code, computer readable instructions, data structures, program modules, and other data for the computer 301. For example and not meant to be limiting, the mass storage device 304 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 304, including by way of example, the operating system 305 and the triage diagnostic aid software 306. Each of the operating system 305 and the triage diagnostic aid software 306 (or some combination thereof) can comprise elements of the programming and the triage diagnostic aid software 306. The triage diagnostic aid data 307 can also be stored on the mass storage device 304. The triage diagnostic aid data 307 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 301 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 303 via the human machine interface 302 that is coupled to the system bus 313, but can be connected by other interface and bus structures, such as a parallel port, game port, an IEEE 1394 Port (also known as a Firewire port), a serial port, or a universal serial bus (USB).

In yet another aspect, the display device 311 can also be connected to the system bus 313 via an interface, such as the display adapter 309. It is contemplated that the computer 301 can have more than one display adapter 309 and the computer 301 can have more than one display device 311. For example, the display device 311 can be a monitor, an LCD (Liquid Crystal Display), or a projector. In addition to the display device 311, other output peripheral devices can comprise components such as speakers (not shown) and a printer (not shown) which can be connected to the computer 301 via the Input/Output Interface 310. Any step and/or result of the methods can be output in any form to an output device. Such output can be any form of visual representation, including, but not limited to, textual, graphical, animation, audio, tactile, and the like. The display device 311 and computer 301 can be part of one device, or separate devices.

The computer 301 can operate in a networked environment using logical connections to one or more remote computing devices 314a,b,c. By way of example, a remote computing device can be a personal computer, portable computer, smartphone, a server, a router, a network computer, a peer device or other common network node, and so on. Logical connections between the computer 301 and a remote computing device 314a,b,c can be made via a network 315, such as a local area network (LAN) and/or a general wide area network (WAN). Such network connections can be through the network adapter 308. The network adapter 308 can be implemented in both wired and wireless environments. Such networking environments are conventional and commonplace in dwellings, offices, enterprise-wide computer networks, intranets, and the Internet.

For purposes of illustration, application programs and other executable program components such as the operating system 305 are illustrated herein as discrete blocks, although it is recognized that such programs and components reside at various times in different storage components of the computing device 301, and are executed by the one or more processors 303 of the computer. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data.

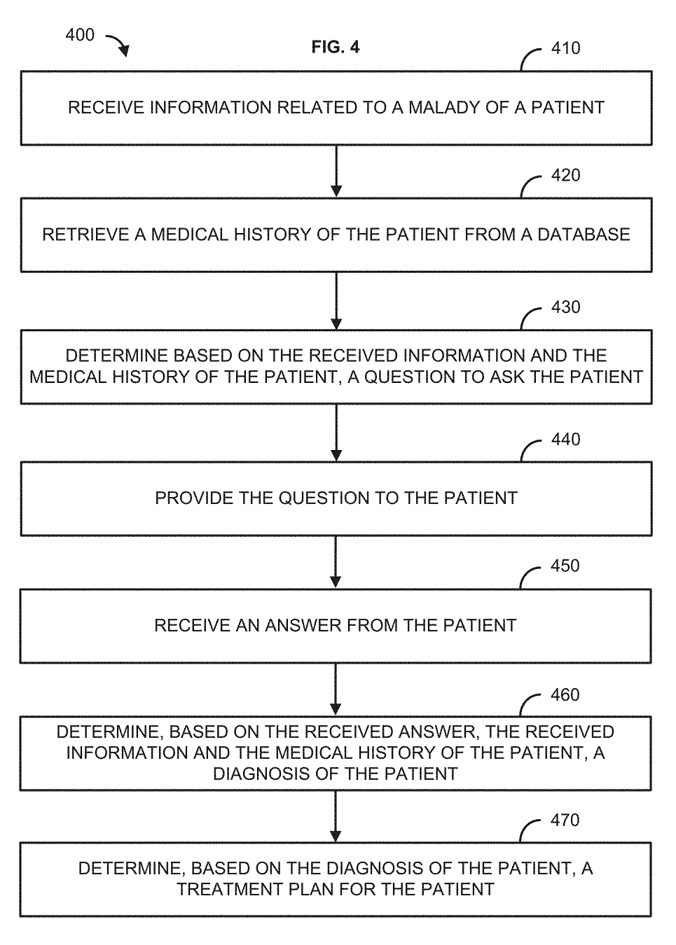

FIG. 4 is a flowchart 400 of an example method. At block 410, information is received related to a malady of a patient. For example, the computing device 102 and/or the QA processing device 110 of FIG. 1A can receive the information.

At block 420, a medical history of the patient is retrieved from a database. For example, the computing device 102 and/or the QA processing device 110 of FIG. 1A can retrieve the medical history from the health care provider database 106.

At block 430, a question to ask the patient based on the received information and the medical history is determined. For example, the computing device 102 and/or the QA processing device 110 of FIG. 1A can determine the question to ask the patient.

At block 440, the question is provided to the patient. For example, the computing device 102 and/or the QA processing device 110 of FIG. 1A can provide the question to the patient.

At block 450, an answer to is received from the patient. For example, the computing device 102 and/or the QA processing device 110 of FIG. 1A can receive the answer from the patient.

At block 460, a diagnosis of the patient is determined based on the received answer, the received information, and the medical history of the patient. For example, the computing device 102 and/or the QA processing device 110 of FIG. 1A can determine the diagnosis of the patient.

At block 470, a treatment plan for the patient is determined based on the diagnosis of the patient. For example, the computing device 102 and/or the QA processing device 110 of FIG. 1A can determine the treatment plan.

In certain aspects, described herein is a method for triaging patients in an emergency room. The method comprises receiving at a first location a first information describing a first state of a patient at a first time. In some aspects the step of receiving the first information comprises conducting an interview with the patient, a caregiver, or any person having knowledge of the patient's medical condition. In other aspects, the step of receiving the first information can further comprise a physical examination of the patient by a medical professional. In some aspects, the first information comprises a chief complaint or a symptom or a group of symptoms. In yet other aspects, the first information can further comprise a sex and an age of the patient. In still further aspects, the first information can comprise a past medical history.

In other aspects, the method disclosed herein further comprises a step of processing the first information to determine a first protocol comprising a first content, wherein the first protocol is provided by a medical provider. In certain aspects, this step of processing is performed by a medical professional to review the first information provided by the patient or caregiver. In certain aspects, the first content of the first protocol comprises a collection step selected from the group consisting of a physical observation by a medical professional, collection of vital signs, blood analysis, imaging, and any combination thereof. In other aspects, the collection step determines the first content of the first protocol.

In further aspects, the first protocol can further comprise entering into a computerized system at least one parameter selected from the group consisting of physical observation by a medical professional, collection of vital signs, blood analysis, imaging, and any combination thereof.

In still further aspects, the method comprises determining based on the first protocol comprising the first content whether the patient is assigned a level I triage and providing a first treatment plan if a level I triage assigned. It is understood that if a level I triage is assigned, the patient is immediately treated by a medical professional. It is understood that the first treatment plan comprises step necessary for a specific patient's medical condition. In certain aspects, if necessary the first treatment plan can further comprise a treatment by a specialist.

In other aspects, where the level I triage is not assigned, the method further comprises moving the patient to a second location. It is understood that the first and the second locations can be any location in the emergency room. It is further understood that the first and the second locations can be the same or different. In the aspects, where the patient is moved to the second location, the method further comprises receiving a second information describing a second state of a patient at a second time.

In some aspects, the second information comprises a data obtained from the first protocol, for example, blood test analysis, imaging results, and like, the patient's chief complaints, or any combination thereof. In some aspects, the step of receiving of the second information is conducted by the disclosed system as describe above. More specifically, in some aspects, the step of receiving the second information can be conducted by the computing device that is in communication with a) the patient and/or the caregiver, and the medical care professional; b) the question-answering processing device; and c) the health care provider database. It is further understood that the question-answering processing device can be also in direct communication with the health care provider database, or a world wide web, or a combination thereof. It is further understood that any step of collection information from the patient or caregiver can be conducted in a language chosen by the patient or the caregiver. In some aspects, the computing device and the question-answering processing device are configured to adjust language setting based on the patient or caregiver preference.

In some aspects the computing device as described above is configured to receive the second information describing the second state of the patient at the second time and to provide this information to the question-answering processing device. In some aspects, the method can further comprise the question-answering processing device to conduct a life-time questioning of the patient and/or caregiver using a set of scripted questions to receive answers and to analyze the answers provided by the patient and/or caregiver to elucidate the nature of a patient risk and to generate a third information.

In other aspects, the method further comprises a step of receiving an electronic health record of the patient, if present. In these aspects, the computing device and/or question-answering processing device can communicate with an entity hosting health records. In some aspects, the entity hosting health records is the healthcare provider database. In other aspects, the entity hosting health records is an external entity. In these aspects, the computing device can comprise an interface connecting the computing device and/or question-answering processing device to the external entity to extract the patient's health records if present. In the aspects, where health records are not present the computing device can optionally create a new patient's health record.

In some aspects, the method further comprises processing the second information, the third information and the electronic health record if present is to determine the first protocol comprising a second treatment plan. In some aspects, the second treatment plan comprises a step of reevaluating a triage level assigned to the patient. If the triage level is reassigned to level I, the patient is immediately treated by a medical professional. If the triage level is not reassigned, the method can further comprise a step of receiving a second protocol having a first content, wherein the second protocol is a worldwide web protocol, and wherein the first content of the second protocol comprises a third treatment plan based on the second state of the patient. In these aspects, the computing device and/or question-answering processing device are in direct communication with the worldwide web and are capable of searching faster than a human. In some aspects the first content of the second protocol can comprise research data, database of updated medical regulations, image database, case studies database or any combination thereof.

In other aspects, the method further comprises a step of optionally comparing the second and the third treatment plans based on the second state of the patient, the second information, the third information and the electronic health record to provide a report for reviews by a medical professional. In these aspects, if the medical professional finds that third treatment plan provides valuable treatment option, the method further comprises adjusting the first protocol comprising the second content to incorporate the first content of the second protocol.

In further aspects, the method further comprises assessing a health risk to the patient based on the first protocol comprising the second content, the second protocol comprising the first content if present, or a combination thereof to provide a health risk assessment of the patient. In certain aspects, the method further comprises an additional step of reevaluating triage level of the patient based in the health risk assessment to determine whether level I triage should be assigned; if level I triage is assigned the patient is moved to the first location to administer the first treatment plan. In the aspect, wherein the level I triage is not assigned, the method further comprises adjusting the first protocol comprising the second content and the second protocol comprising the first content, if present, based on the health risk assessment to provide a fourth treatment plan. In some aspects, it is understood that the health risk assessment comprises analysis of the second information, third information, the health records, medical professional observations, or a combination thereof. It is further understood that the reevaluation of the triage level can be done at any step of the disclosed method.

In some other aspects, the second, the third and the fourth treatment plans can comprise any treatments available for a specific patient medical condition. In some unlimiting examples, the second, the third, and the fourth plans can include additional blood test analysis, imaging, consultation with a specialist, a system specific treatment or testing, administration of medications, and the like.

In further aspects, the method further comprises a generating a report based on the first information, second information, third information, the first treatment plan, the second treatment plan, the third treatment plan, and the fourth treatment plan, or a combination thereof for review by a medical professional.

In certain aspects, the method described herein further comprises generating a prescription. In some aspects, the prescription is generated by the computer device based on the first, the second, the third or the treatment plan. It is understood that the generated prescription is reviewed by the medical professional prior to administrating the medication to the patient. In yet other aspects, the method further comprises admitting the patient into a medical provider facility. In these aspects, the computing device based on the first, second, third or fourth treatment plan proposes to admit the patient to the medical provider facility. The proposal is reviewed by the medical professional and if appropriate the patient is admitted. In yet other aspects, the method can comprise discharging the patient from the medical provider facility. In these aspects, the computing device based on the first, second, third or fourth treatment plan proposes to discharge the patient to the medical provider facility. The proposal is reviewed by the medical professional and if appropriate the patient is discharged.

In still further aspects, the method disclosed herein further comprises generating a billing report. In these aspects, the computing device can collect all billing information related to all administered test and treatment plans and create a billing report to be submitted to an insurance company, the patient, or the caregiver.

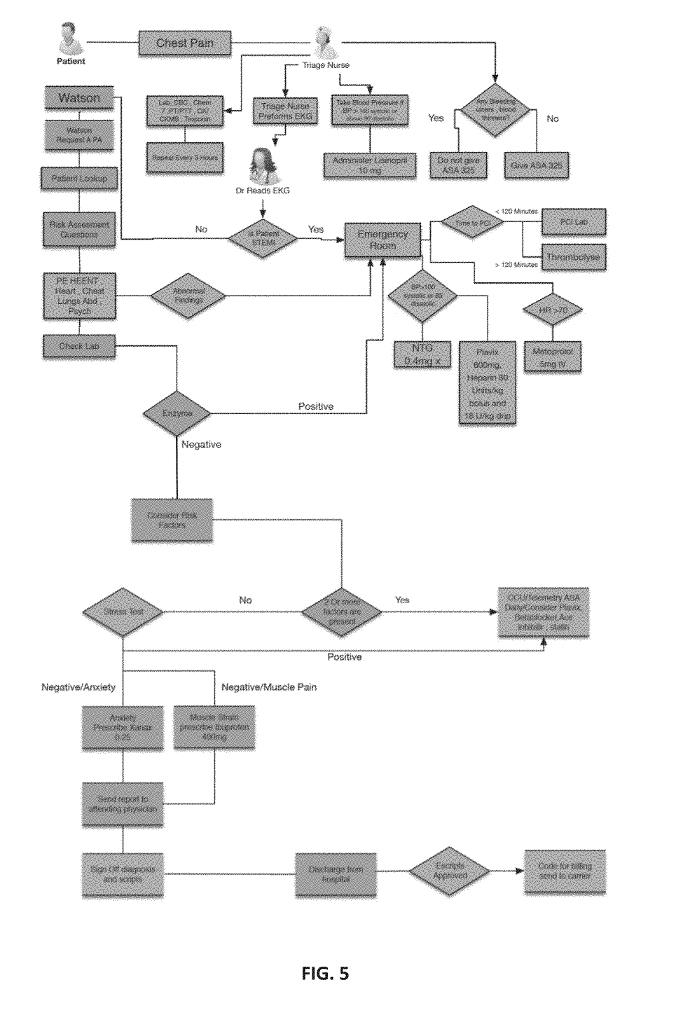

An exemplary method for triaging patients complaining on a chest pain in an emergency room comprising is demonstrated in FIG. 5. In certain aspects, the patient or a caregiver with a chief complaint of chest pain arrives in an emergency room. The method comprises receiving at a first location first information provided by the patient of the caregiver and describing a first state of the patient at a first time. The step of receiving can be conducted by a triage medical profession, for example nurse, a doctor, or a physician assistant. It is understood that the first information comprises a chief complaint, a sex and an age of the patient, a health history, or a combination thereof. The method further comprises processing this information to determine a first protocol related to a chest pain comprising a first content, wherein the first protocol is provided by a medical provider. Specifically, in this exemplary aspect, the first content of the first protocol can comprise a request for a blood laboratory analysis for a complete blood count (CBC), a Chem-7 test to evaluate kidney function, blood acid/base balance, levels of blood sugar and electrolytes, a prothrombin time/a partial thromboplastic time (PT/PTT) test to evaluate a possible bleeding or clotting and to evaluate hemostasis, a creatine kinase-MB (CK-CKMB) test to evaluate an amount of creatine kinase in the blood to determine is there any heart damage or skeletal damage possible, and a troponin test to measure an amount of troponin to differentiate between unstable angina and myocardial infarction. In some aspects, this blood laboratory analysis can be repeated every three hours. Further, in the exemplary aspect, the first content of the first protocol can comprise an EKG measurement, collecting of vital signs such as a heart rate, blood pressure, and temperature. In still further aspects, the doctor can review the data obtained by administering the first protocol comprising a first content. If the findings indicate that the patient may experience a ST-elevation myocardial infarction, the patient is assigned a level I triage and a first treatment plan is provided. In these unlimiting exemplary aspects, the first treatment can comprise administrating a primary percutaneous coronary intervention (PCI). In some aspects, if the time to PCI is less than 120 minutes, the patient is transferred to PCI lab and PCI is performed. In other aspects, is the time to PCI is more than 120 minutes, the patient can undergoes thrombolysis.

The first treatment plan can further comprise administrating of medications depending on a blood pressure and a heart rate.

In further aspects, the first protocol further comprises entering into a computerized system at least one parameter selected from the group consisting of physical observation by a medical professional, collection of vital signs, blood analysis, imaging, and any combination thereof.

In the aspects where the level I triage is not assigned, the patient can be moved to a second location. It is understood that the first and the second locations can be the same or different. At the second location the patient can interact with the disclosed above disclosed system. In some aspect, the patient and/or caregiver are in communication with the computing device capable of receiving a second information describing a second state of a patient at a second time. More specifically, in some aspects, the step of receiving the second information can be conducted by the computing device that is in communication with a) the patient and/or the caregiver, and the medical care professional; b) the question-answering processing device; and c) the health care provider database. It is further understood that the question-answering processing device can be also in direct communication with the health care provider database, or a world wide web, or a combination thereof. In some aspects, the computing device and the question-answering processing device are configured to adjust language setting based on the patient or caregiver preference.

In some aspects, the second information comprises a data obtained from the first protocol, for example, blood test analysis, imaging results, and like, the patient's chief complaints, or any combination thereof.

In the exemplary aspect, the question-answering processing device is IBM-Watson™. However, it is understood that any question-answering processing device capable of cognitive computing can be utilized. In some aspects the computing device as described above is configured to receive the second information describing the second state of the patient at the second time and to provide this information to the question-answering processing device.

In other exemplary aspects, the question-answering processing device and/the computing device receives an electronic health record if present from the health care provider database. In the aspects, where the health care provider does not have the patient's electronic health record, the disclosed system can communicate with any entity hosting health records. In yet other aspects, the disclosed system can initiate new health records associated with the patient.

In some exemplary aspects, the second content of the first protocol can further comprise a step of a physical observation by a medical professional to determine whether a level II triage or a level III triage is assigned to the patient. In these exemplary aspects, the question-answering processing device can request a presence of a medical professional, for example, a physician assistant or a nurse to observe the patient.

In still further aspects, the method comprises processing the second information and the electronic health record if present to determine a first protocol comprising a second content, wherein the second content comprises a second treatment plan. In these aspects, the second treatment is based on results obtained in the step of the physical observation by a medical professional and based on whether the patient is assigned level II or level III triage.

In yet other aspects, the disclosed system can optionally (not shown on FIG. 5) receive a second protocol related to a chest pain having a first content, wherein the second protocol is a worldwide web protocol, and wherein the first content of the second protocol comprises a third treatment plan based on the second state of the patient. In some exemplary aspects, the method can further optionally comprise a step of comparing the second and the third treatment plans based on the second state of the patient and the electronic health record of the patient to provide a report for review by a medical professional. In still further aspects, the method can further comprise optionally adjusting the first protocol to incorporate the first content of the second protocol. In some aspects, the first content of the second protocol comprises research data, database of updated regulations, image database, case studies database or any combination thereof. In certain aspects, the third treatment is based on the second information, results obtained in the step of physical observation by a medical professional, and data obtained from the worldwide web.

In still further aspects, the method can further comprise assessing a health risk to the patient based on the first protocol comprising the second content, the second protocol comprising the first content if present, or a combination thereof to provide a health risk assessment and to provide a third information. In yet other aspects, the risk assessment is performed by the question-answering processing device capable of conducting a life-time questioning of the patient and/or caregiver using a set of scripted questions to receive answers and to analyze the answers provided by the patient and/or caregiver to elucidate the nature of a patient risk and to generate a third information. In other aspects, the first protocol comprising the second content and the second protocol comprising the first content, if present, based on the health risk assessment to provide a fourth treatment plan. In yet other aspects, the fourth treatment plan is administered.

In some exemplary aspects, the fourth treatment plan can comprise a physical examination of head, eyes, ears, nose and throat, together with examination of heart, chest, lungs, abdominal, and psychological analysis of the patient. In some aspects, the fourth treatment can further comprise a step of reassigning the triage level based on the second information, health records, first protocol, second protocol, risk assessment, or a combination thereof. For example, the triage level of the patient can be re-evaluated based on the fourth treatment and if abnormal findings are found, the level I triage is re-assigned and the patient is moved to the first location to administer the first treatment plan.

The fourth treatment can further comprise an additional blood laboratory analysis. In some exemplary aspects, the fourth treatment requires to monitor the blood for a presence of a cardiac enzyme. In exemplary aspects, where enzyme is present, the level of triage is reassigned to level I triage and the patient is moved to the first location and the first treatment plan is administered.

In yet other exemplary aspects, where the cardiac enzyme is not observed in the blood analysis, the health risk assessment is reviewed by the disclosed system for a presence of at least two risk factors. In the aspects, where at least two risk factors are present, the method comprises administering to the patient a fifth treatment plan provided by the first protocol, the second protocol if present, or a combination thereof. In some aspects, the fifth treatment plan comprises administrating a medication. It is understood that while the disclosed system is proposing the medications prescribed by the first protocol, the second protocol if present, the proposed medications are reviewed by the medical professionals and administered by the medical professionals. In some aspects, the medications can comprise beta blockers, ace inhibitors, statin, and the like.

In the aspects, where less than two risk factors are present, the method comprises administering to the patient a sixth treatment plan provided by the first protocol, the second protocol if present, or a combination thereof. In some aspects, the sixth treatment plan can comprise a stress test. In the aspects, where results of the stress test are positive, the disclosed system will require a medical professional to administer the fifth treatment plan. In the aspect, where results of the stress test are negative the method comprises administering a seventh treatment plan. In some aspects, the seventh treatment plan comprises administering at least one medication. In some aspects, the medications can comprise anxiety medications or medication addressing muscle strains. It is understood that all proposed by the system treatments and medications are reviewed by the medical professionals.

In further aspects, the method comprises generating a report based on the first information, second information, third information, the first treatment plan, the second treatment plan, the third treatment plan, the fourth treatment plan, the fifth treatment plan, the sixth treatment plan, the seventh treatment plan or a combination thereof for review by a medical professional. In still further aspects, the method comprises generating prescriptions and proposing a final diagnosis for a review by a medical professional. In still further aspects, the method comprises admitting the patient into a medical provider facility if the final diagnosis requires it. In yet other aspects, the method comprises discharging the patient from a medical provider facility if the final diagnosis requires it.

In till further aspects, the method disclosed herein further comprises generating a billing report. In these aspects, the computing device can collect all billing information related to all administered test and treatment plans and create a billing report to be submitted to an insurance company, the patient, or the caregiver.

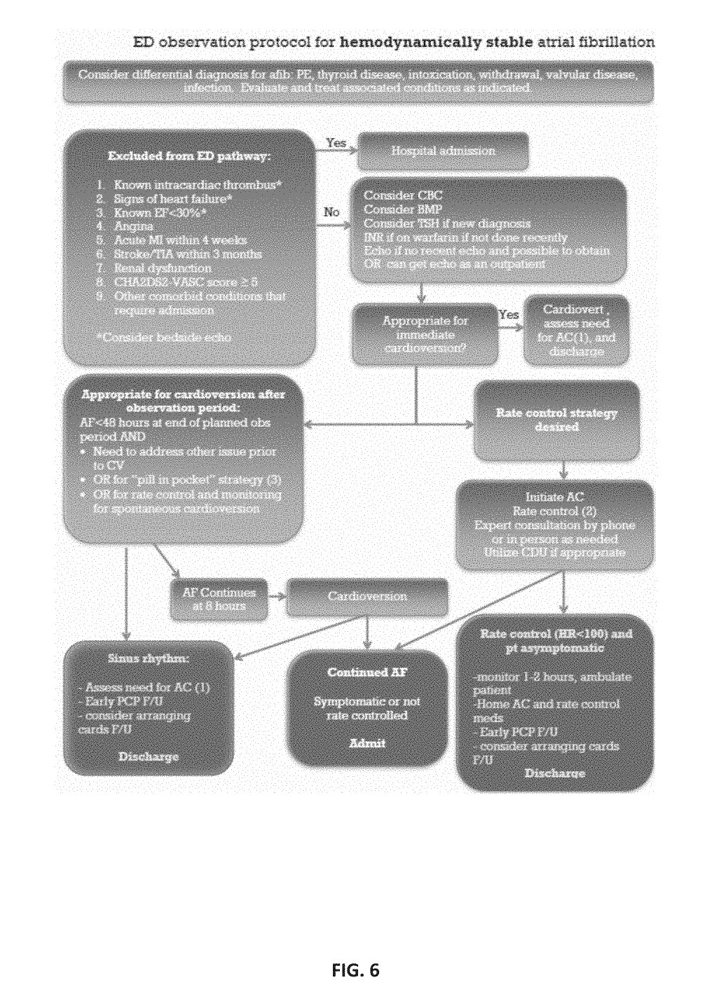

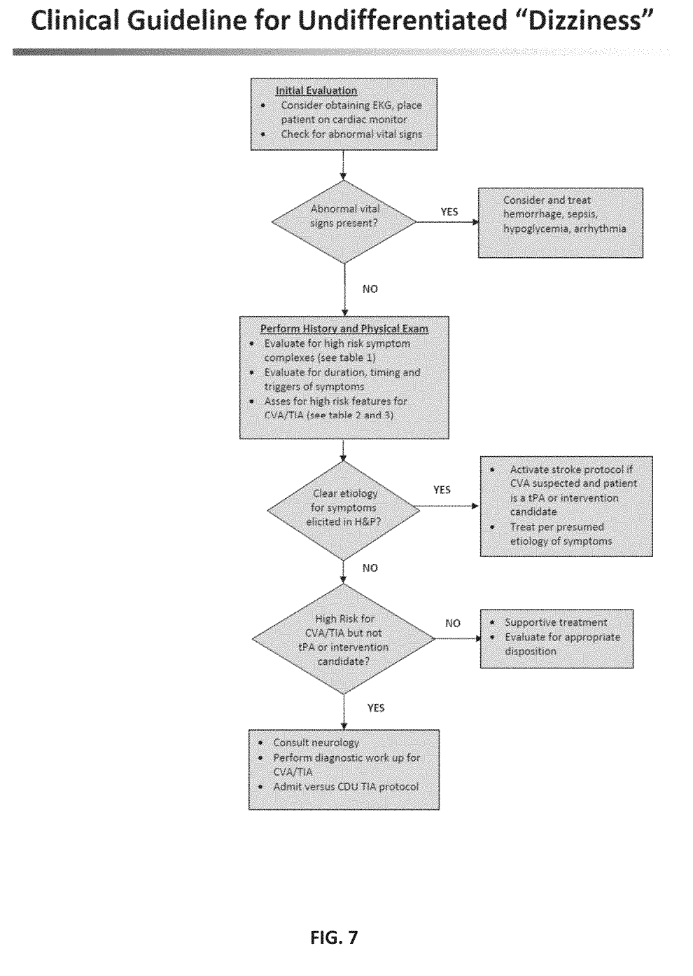

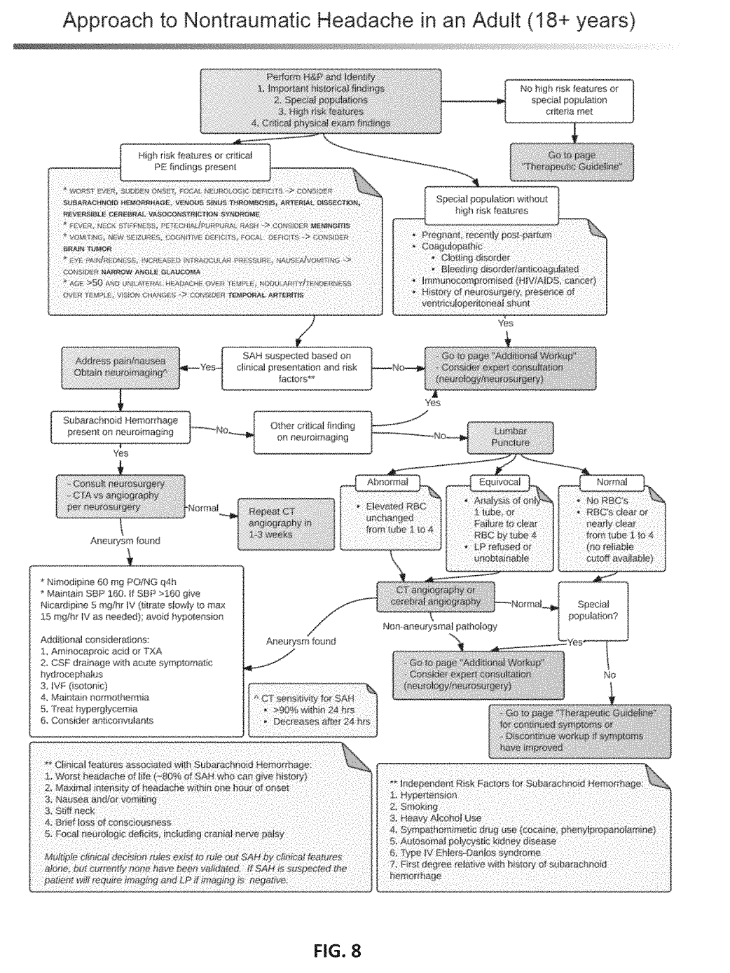

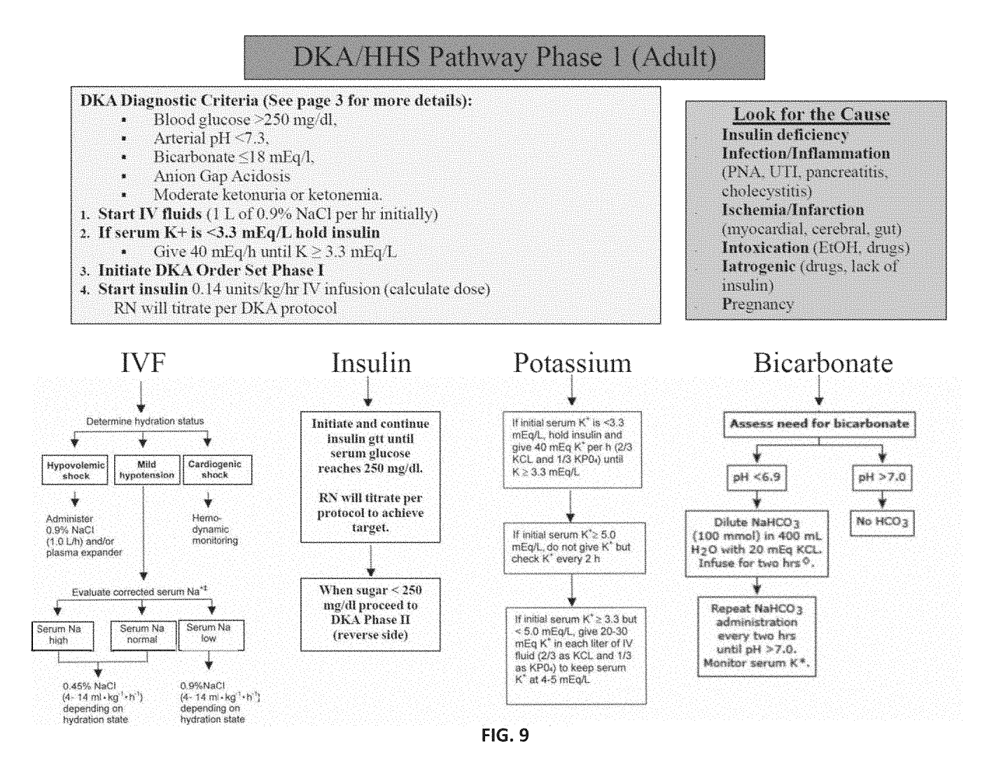

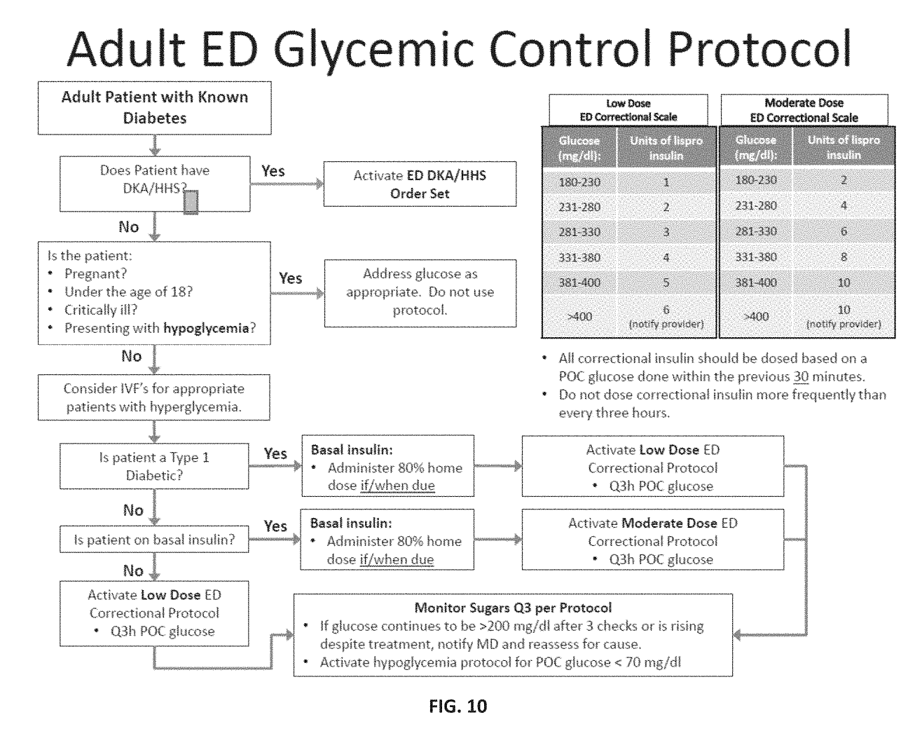

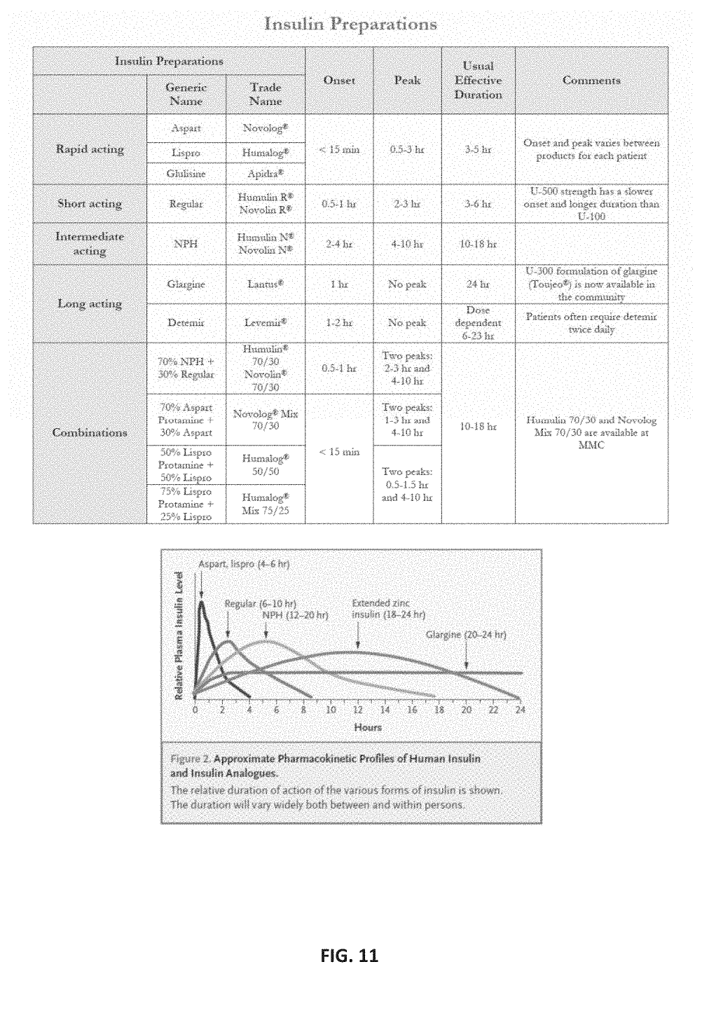

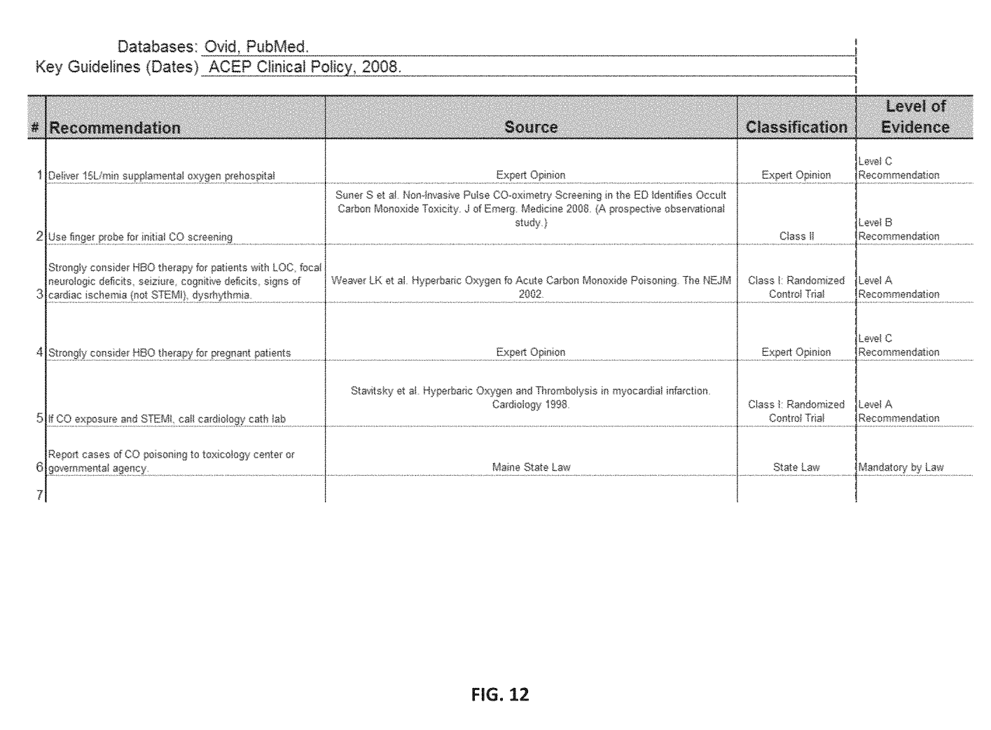

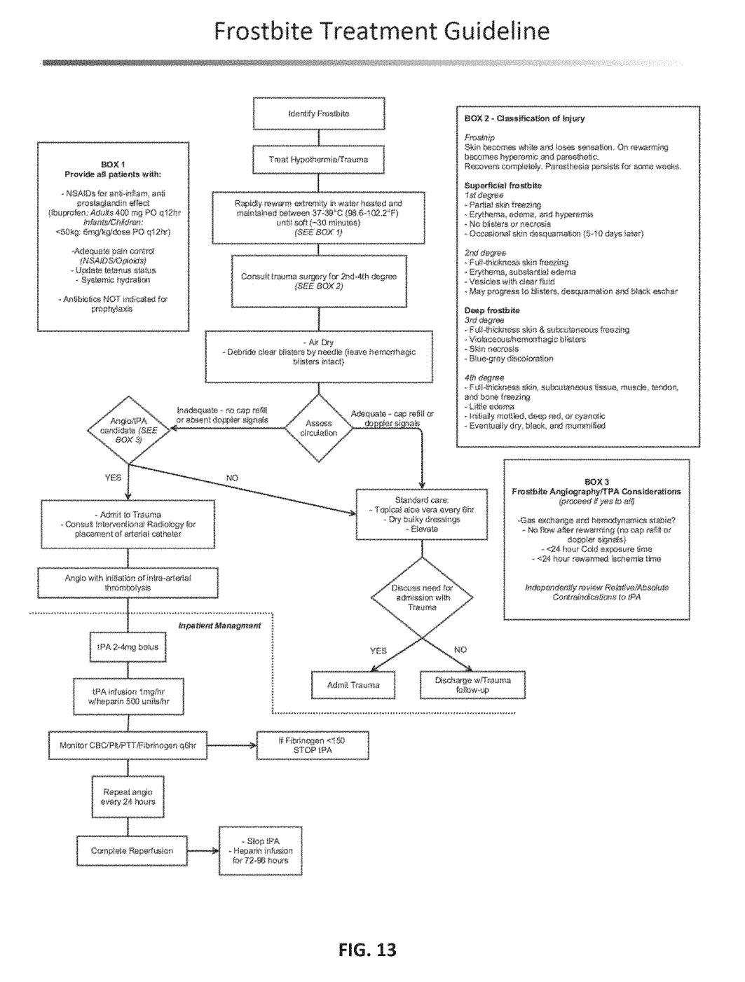

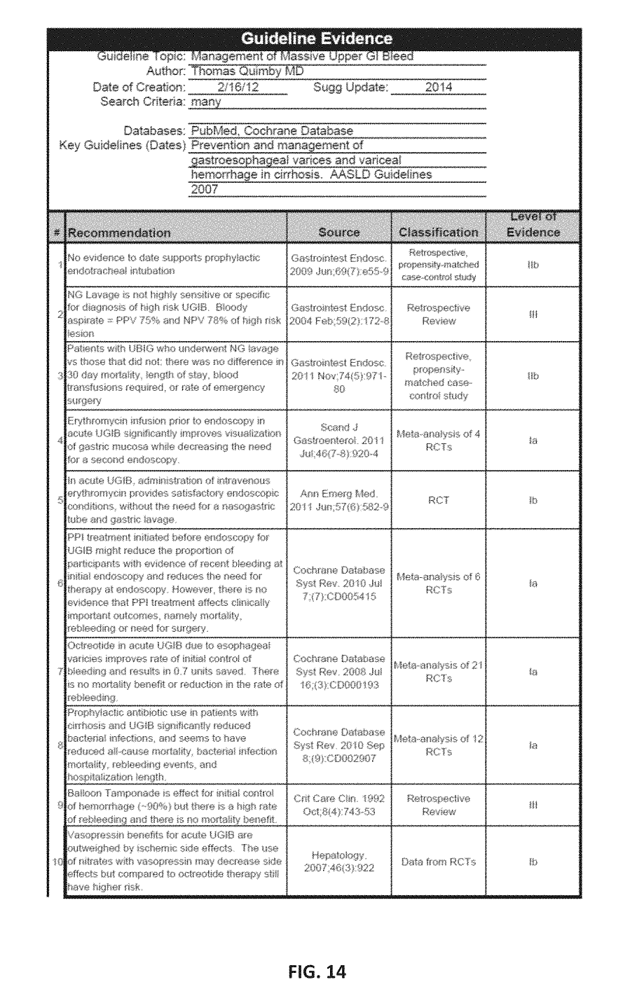

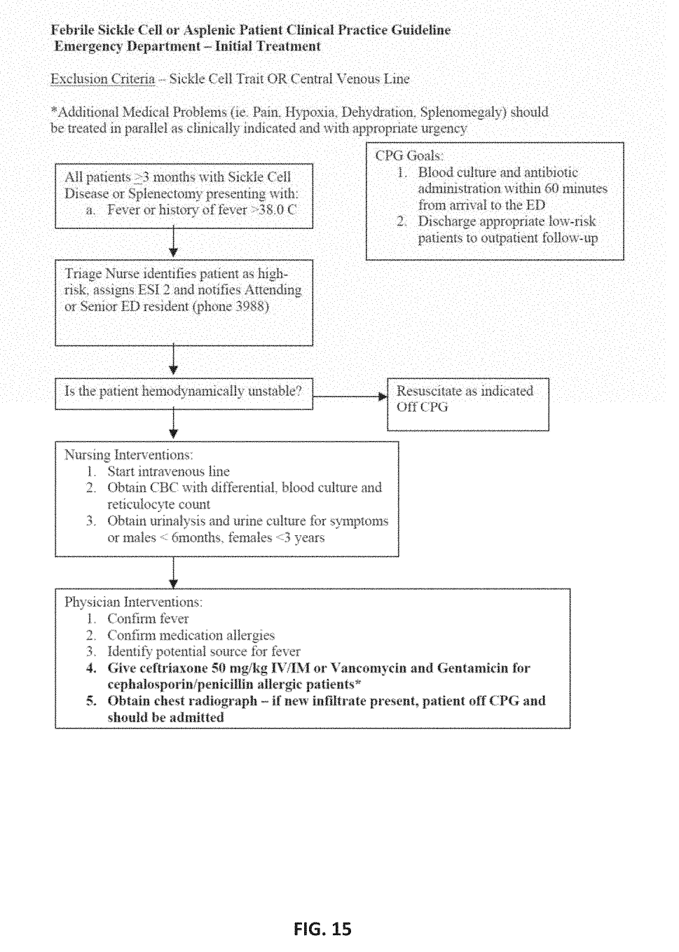

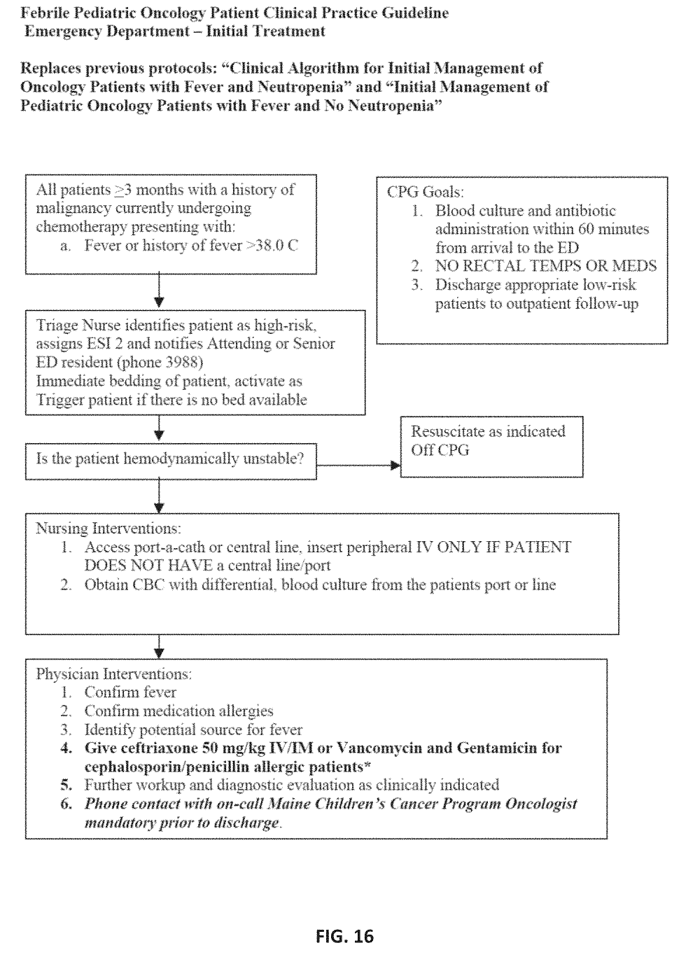

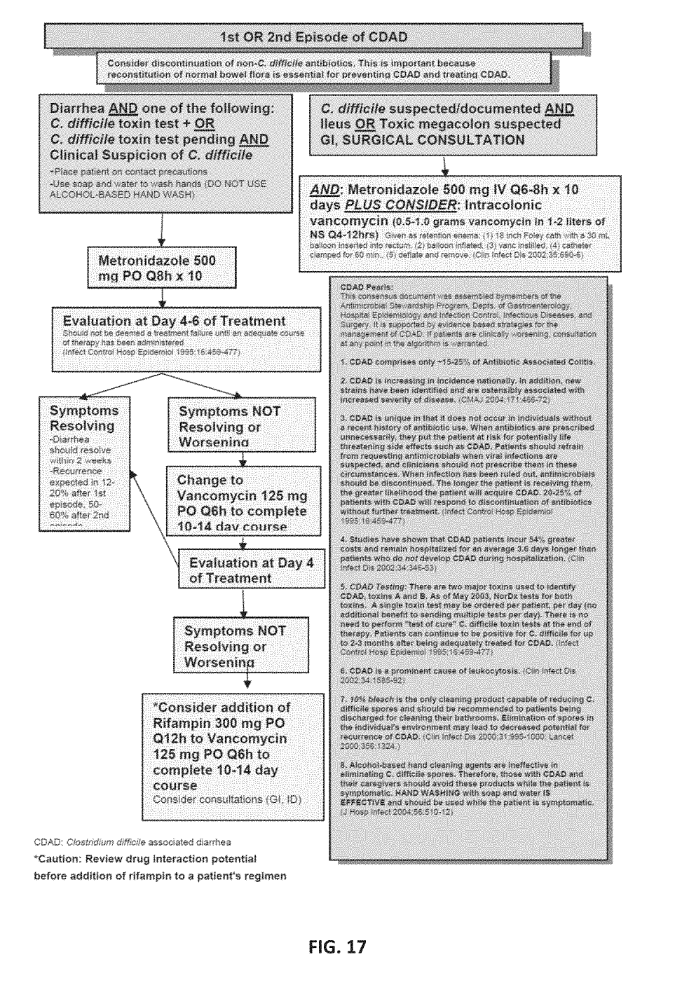

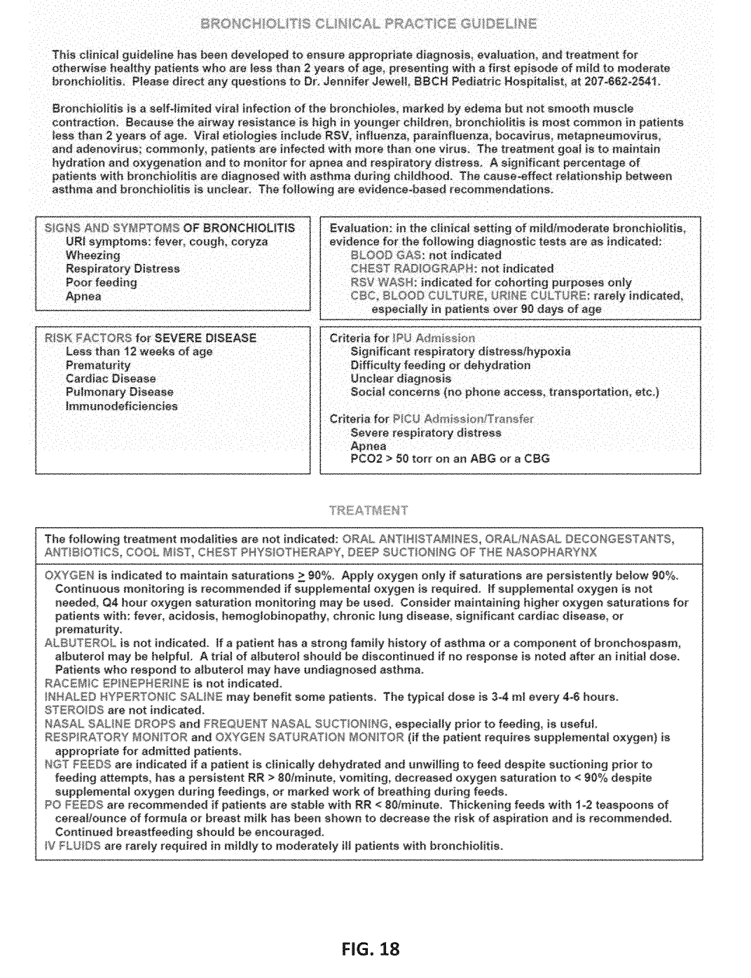

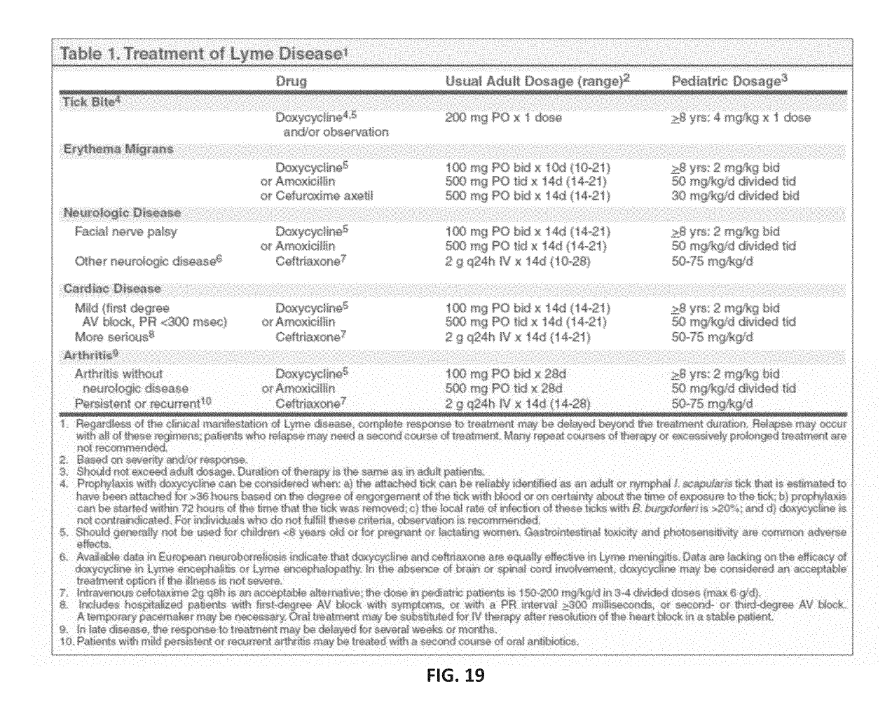

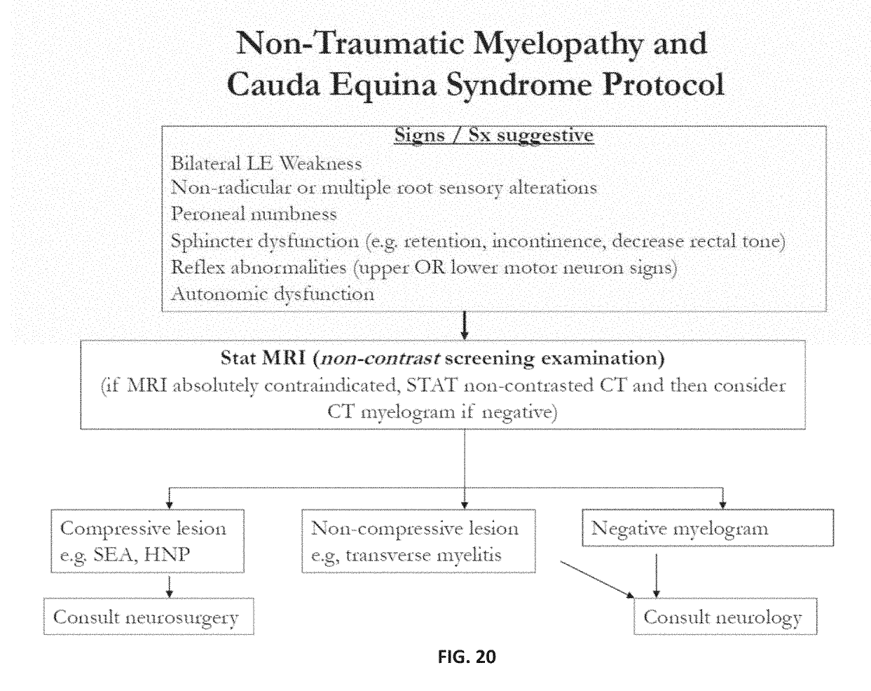

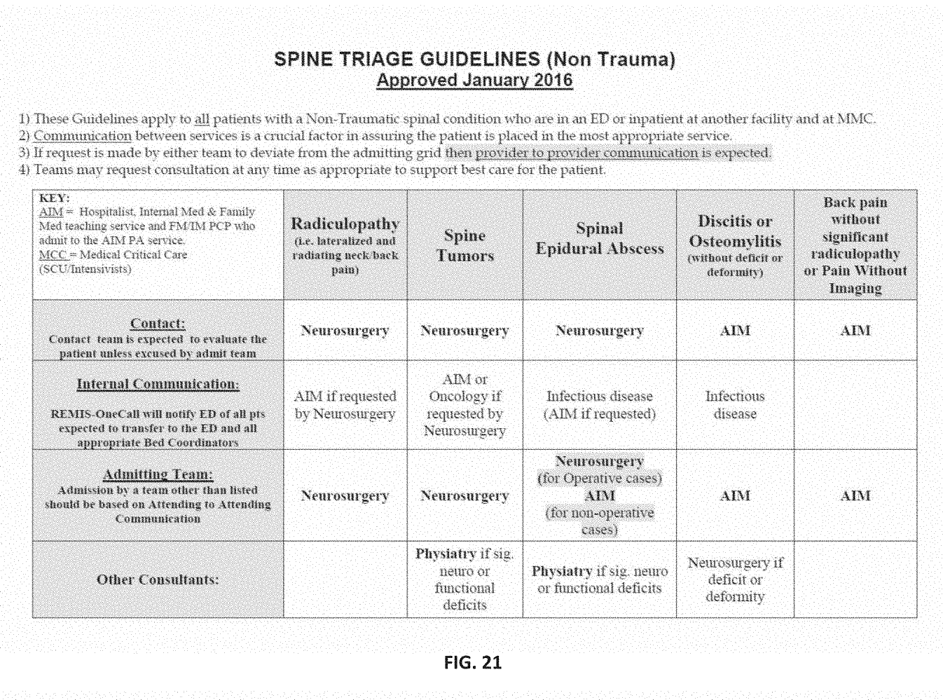

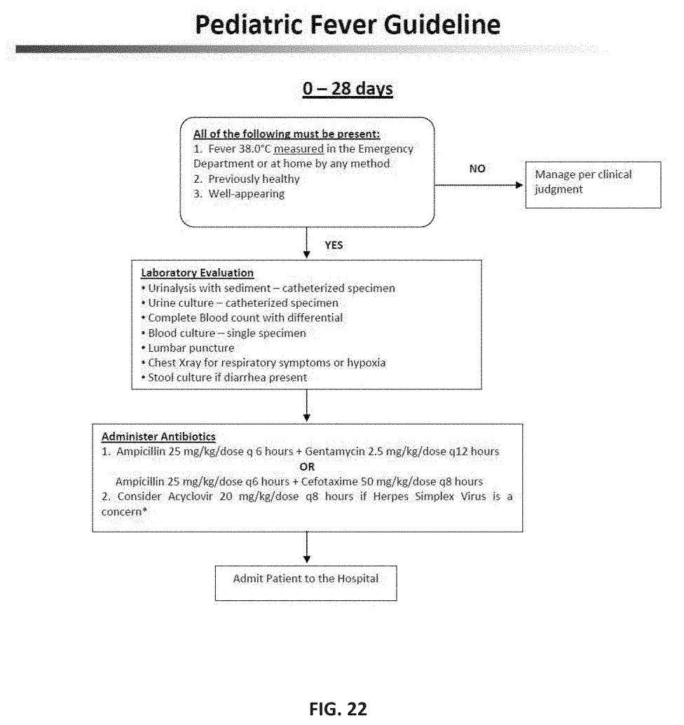

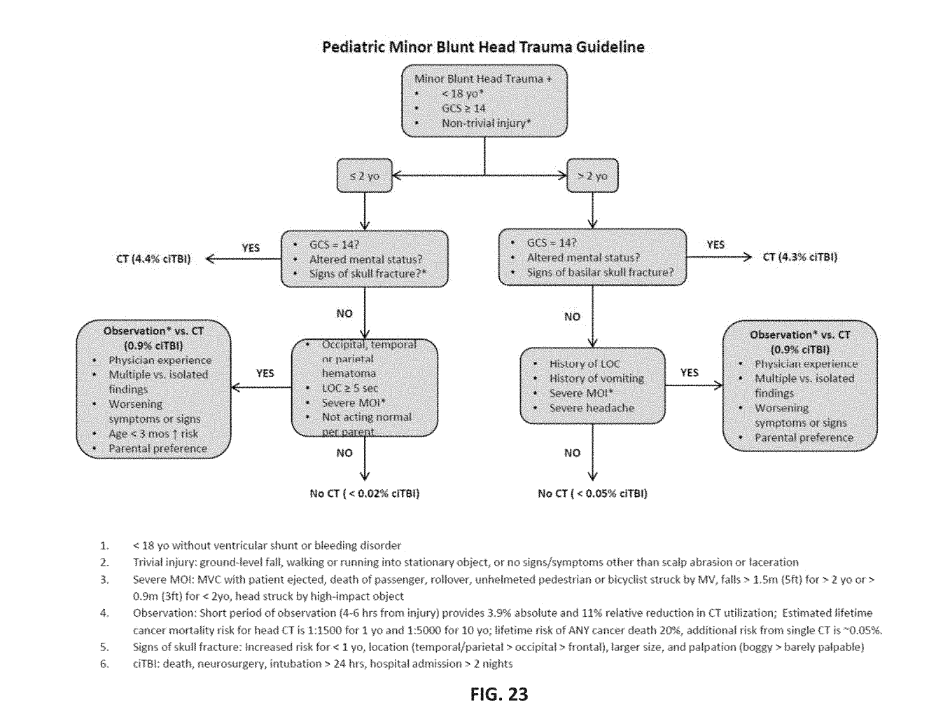

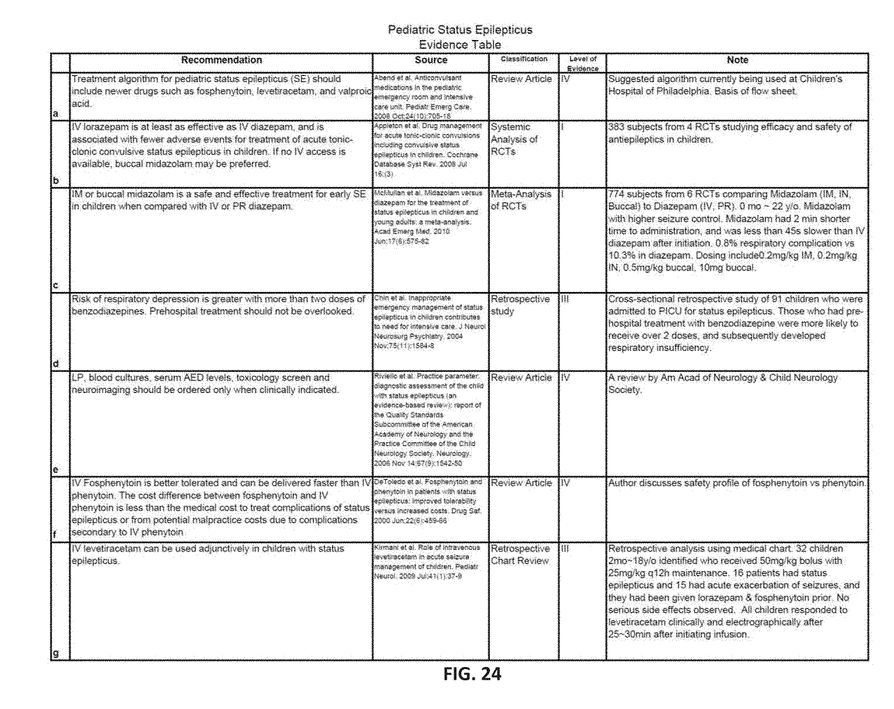

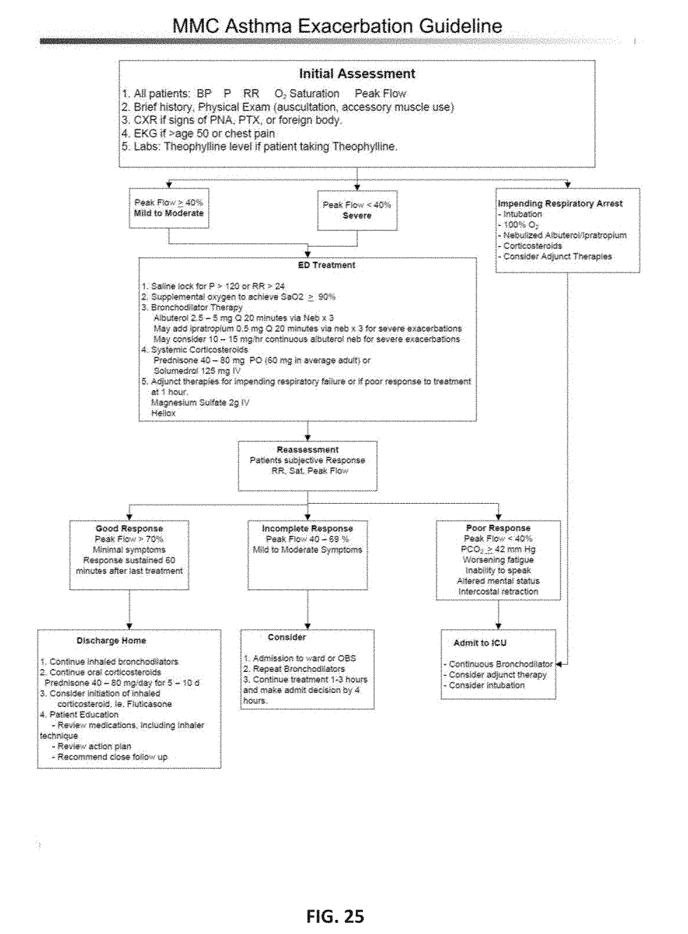

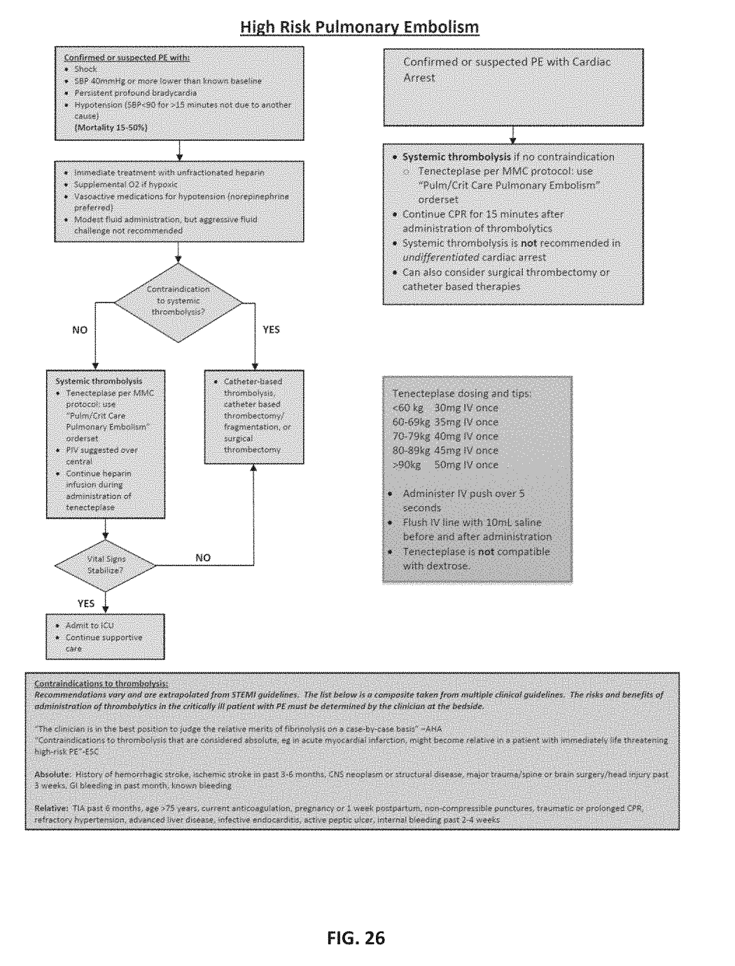

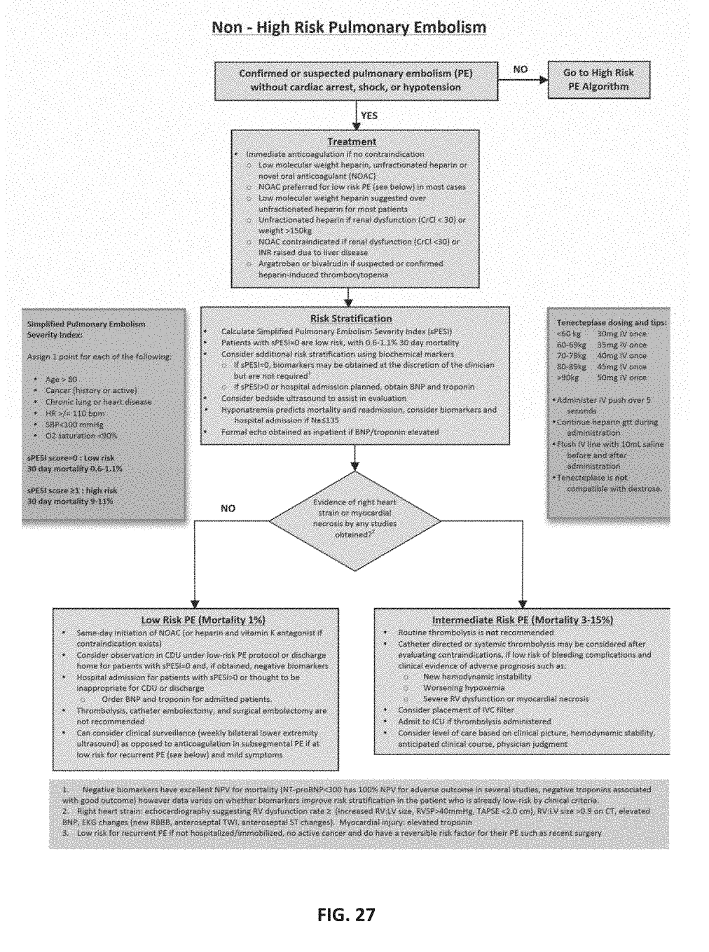

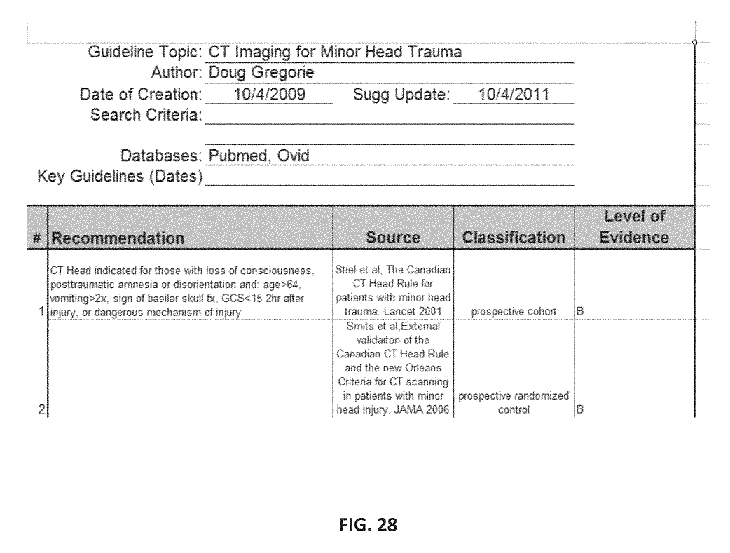

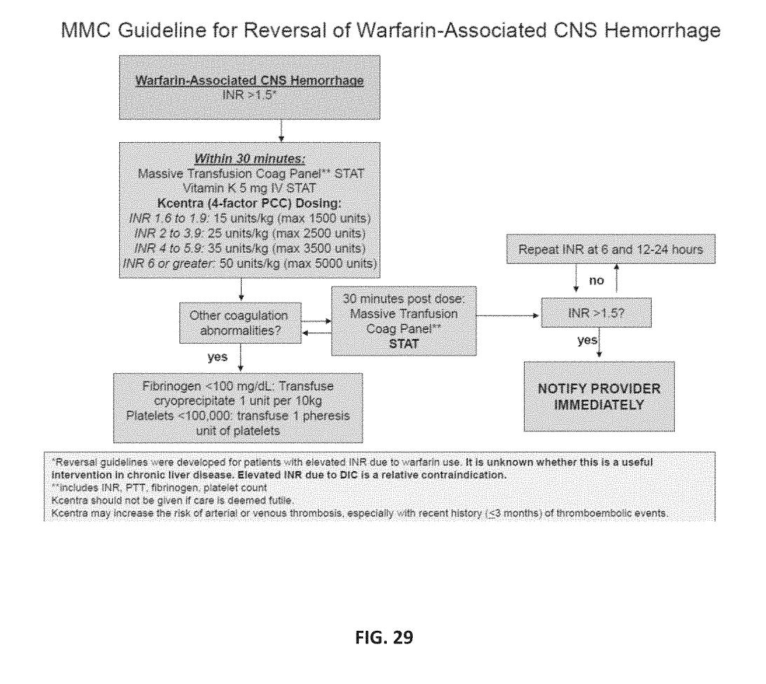

FIGS. 6-29 show additional exemplary protocols that can be practiced by the disclosed systems and disclosed methods. In some exemplary aspects, the systems and methods disclosed herein can be applied to a protocol showing ED observation protocol for hemodynamically stable atrial fibrillation (FIG. 6). In other exemplary aspects, the systems and methods disclosed herein can be applied to a protocol for undifferentiated “dizziness.” (FIG. 7). In other exemplary aspects, the system and method disclosed herein can be applied to a protocol showing the approach to nontraumatic headache in an adult (18+ years) (FIG. 8). In other exemplary aspects, the systems and methods disclosed herein can be applied to a protocol showing DKA (Diabetic Ketoacidosis)/HHS (Hyperosmolar Hyperglycemic State) pathways in an adult (FIG. 9). In other exemplary aspects, the systems and methods disclosed herein can be applied to a protocol Adult ED glycemic control protocol (FIG. 10). In other exemplary aspects, the systems and methods disclosed herein can be applied to a protocol showing insulin preparations (FIG. 11). In other exemplary aspects, the systems and methods disclosed herein can be applied to a protocol showing guideline evidence of carbon monoxide poisoning (FIG. 12). In other exemplary aspects, the systems and methods disclosed herein can be applied to a protocol showing a guideline for frostbite treatment (FIG. 13). In other exemplary aspects, the systems and methods disclosed herein can be applied to a protocol showing management of massive upper GI bleed. (FIG. 14). In other exemplary aspects, the systems and methods disclosed herein can be applied to a protocol showing febrile sickle cell or asplenic patient clinical practice guideline emergency department—initial treatment (FIG. 15). In other exemplary aspects, the systems and methods disclosed herein can be applied to a protocol showing febrile pediatric oncology patient clinical practice guideline emergency department—initial treatment (FIG. 16). In other exemplary aspects, the systems and methods disclosed herein can be applied to a protocol showing the treatment for a 1^{st} or 2^{nd} episode of CDAD (Clostridium Difficile-Associated Diarrhea). (FIG. 17). In other exemplary aspects, the systems and methods disclosed herein can be applied to a protocol for bronchiolitis (FIG. 18). In other exemplary aspects, the systems and methods disclosed herein can be applied to a protocol for the treatment of Lyme Disease (FIG. 19). In other exemplary aspects, the systems and methods disclosed herein can be applied to a protocol showing non-traumatic myelopathy and Cauda Equina Syndrome (FIG. 20). In other exemplary aspects, the systems and methods disclosed herein can be applied to a protocol spine triage guidelines (non-trauma) (FIG. 21). In other exemplary aspects, the systems and methods disclosed herein can be applied to a protocol for pediatric fever (FIG. 22). In other exemplary aspects, the systems and methods disclosed herein can be applied to a protocol for pediatric minor blunt head trauma (FIG. 23). In other exemplary aspects, the systems and methods disclosed herein can be applied to a protocol pediatric status epilepticus evidence table (FIG. 24) In other exemplary aspects, the systems and methods disclosed herein can be applied to a protocol showing the MMC asthma exacerbation guideline (FIG. 25). In other exemplary aspects, the systems and methods disclosed herein can be applied to a protocol showing high risk pulmonary embolism (FIG. 26). In other exemplary aspects, the systems and methods disclosed herein can be applied to a protocol showing non-high risk pulmonary embolism (FIG. 27). In other exemplary aspects, the systems and methods disclosed herein can be applied to a protocol showing minor blunt head trauma algorithm (FIG. 28). In other exemplary aspects, the systems and methods disclosed herein can be applied to a protocol showing MMC's guideline for reversal of Warfarin-associated CNS (Central Nervous System) Hemorrhage (FIG. 29).

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the methods and systems.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method, comprising:
receiving, by a computing device from a patient, information related to a malady of the patient;
retrieving, by the computing device from a database storing medical histories of a plurality of patients, a medical history of the patient;
determining, by the computing device based on the received information and the medical history of the patient, a first question to ask the patient;
providing, by the computing device, the first question to the patient;
receiving, by the computing device, from the patient a first answer;
determining, by the computing device based on the received first answer, the received information, and the medical history of the patient, a diagnosis of the patient; and
determining, by the computing device based on the diagnosis of the patient, a treatment plan for the patient.

2. The method of claim 1, wherein the information related to the malady is provided in a human language, wherein the method further comprises determining, by a natural language processor of the computing device, the information related to the malady.

3. The method of claim 1, further comprising determining, by the computing device based on the received information, the medical history of the patient and the received answer, one or more additional questions to ask the patient.

4. The method of claim 1, further comprising, prior to determining the diagnosis of the patient, determining, by the computing device, a hypothesis associated with the diagnosis of the patient, wherein the hypothesis comprises a first possible diagnosis of a plurality of possible diagnosis.

5. The method of claim 1, further comprising determining, by the computing device, a confidence score associated with the diagnosis of the patient, wherein the confidence score is determined based on evidence sources that support the diagnosis.

6. The method of claim 1, wherein the treatment plan is based on the capabilities of a health care provider that will execute the determined treatment plan, wherein the capabilities of the health care provider comprise at least one of medicines, physicians, or facilities associated with the health care provider.

7. The method of claim 1, wherein the computing device is configured to access a diagnostic information database comprising one or more diagnostic procedures based on medical guidelines, the method further comprising learning, by the computing device from the diagnostic information database, the one or more diagnostic procedures to properly diagnose the patient.

8. An apparatus, comprising:
one or more processors; and
a memory storing processor executable instructions that, when executed by the one or more processors, cause the apparatus to:
receive, from a patient, information related to a malady of the patient;
retrieve, from a database storing medical histories of a plurality of patients, a medical history of the patient;
determine, based on the received information and the medical history of the patient, a first question to ask the patient;
provide the first question to the patient;
receive from the patient a first answer;
determine, based on the received first answer, the received information and the medical history of the patient, a diagnosis of the patient; and
determine, by the computing device based on the diagnosis of the patient, a treatment plan for the patient.

9. The apparatus of claim 8, wherein the information related to the malady is provided in a human language, wherein the method further comprises determining, by a natural language processor of the computing device, the information related to the malady.

10. The apparatus of claim 8, wherein the processor executable instructions, when executed by the one or more processors, further cause the apparatus to determine, based on the received information, the medical history of the patient and the received answer, one or more additional questions to ask the patient.

11. The apparatus of claim 8, wherein the processor executable instructions, when executed by the one or more processors, further cause the apparatus to, prior to determining the diagnosis of the patient, determine a hypothesis associated with the diagnosis of the patient, wherein the hypothesis comprises a first possible diagnosis of a plurality of possible diagnosis.

12. The apparatus of claim 8, wherein the processor executable instructions, when executed by the one or more processors, further cause the apparatus to determine a confidence score associated with the diagnosis of the patient, wherein the confidence score is determined based on evidence sources that support the diagnosis.

13. The apparatus of claim 8, wherein the treatment plan is based on the capabilities of a health care provider that will execute the determined treatment plan, wherein the capabilities of the health care provider comprise at least one of medicines, physicians, or facilities associated with the health care provider.

14. The apparatus of claim 8, wherein the processor executable instructions, when executed by the one or more processors, further cause the apparatus to:
access a diagnostic information database comprising one or more diagnostic procedures based on medical guidelines, and
learn, from the diagnostic information database, the one or more diagnostic procedures to properly diagnose the patient.

15. A computer readable medium comprising computer executable instructions configured to cause a computing device to:
receive, from a patient, information related to a malady of the patient;
retrieve, from a database storing medical histories of a plurality of patients, a medical history of the patient;
determine, based on the received information and the medical history of the patient, a first question to ask the patient;
provide the first question to the patient;
receive from the patient a first answer;
determine, based on the received first answer, the received information and the medical history of the patient, a diagnosis of the patient; and
determine, by the computing device based on the diagnosis of the patient, a treatment plan for the patient.

16. The computer readable medium of claim 15, wherein the information related to the malady is provided in a human language, wherein the method further comprises determining, by a natural language processor of the computing device, the information related to the malady.

17. The computer readable medium of claim 15, wherein the computer executable instructions are further configured to cause the computing device to determine, based on the received information, the medical history of the patient and the received answer, one or more additional questions to ask the patient.

18. The computer readable medium of claim 15, wherein the computer executable instructions are further configured to cause the computing device to, prior to determining the diagnosis of the patient, determine a hypothesis associated with the diagnosis of the patient, wherein the hypothesis comprises a first possible diagnosis of a plurality of possible diagnosis.

19. The computer readable medium of claim 15, wherein the computer executable instructions are further configured to cause the computing device to determine a confidence score associated with the diagnosis of the patient, wherein the confidence score is determined based on evidence sources that support the diagnosis.

20. The computer readable medium of claim 15, wherein the treatment plan is based on the capabilities of a health care provider that will execute the determined treatment plan, wherein the capabilities of the health care provider comprise at least one of medicines, physicians, or facilities associated with the health care provider.
